(19) United States
(12) Patent Application Publication
(10) Pub. No.: US 2016/0279299 A1
(43) Pub. Date: Sep. 29, 2016

(54) SILK FIBROIN-BASED FLUID DELIVERY SYSTEMS AND DEVICES

(71) Applicants: TRUSTEES OF TUFTS COLLEGE, Medford, MA (US); UNIVERSITY OF PITTSBURGH - OF THE COMMONWEALTH SYSTEM OF HIGHER EDUCATION, Pittsburgh, PA (US)

(72) Inventors: David L. Kaplan, Concord, MA (US); Eun Seok Gil, Acton, MA (US); J. Peter Rubin, Pittsburgh, PA (US); Jolene E. Valentin, Pittsburgh, PA (US)

(21) Appl. No.: 15/036,910

(22) PCT Filed: Nov. 21, 2014
(86) PCT No.: PCT/US2014/066806

§ 371 Date: May 16, 2016

Related U.S. Application Data
(60) Provisional application No. 61/907,250, filed on Nov. 21, 2013.

(51) Int. Cl. A61L 29/04; A61L 29/00; A61L 31/04; A61L 29/14; A61L 29/16

(52) U.S. Cl. CPC A61L 29/048; A61L 29/049; A61L 29/146; A61L 29/16; A61L 29/148; A61L 31/047; A61L 29/005; A61L 2300/252; A61L 2300/222; A61L 2300/416; A61L 2300/412; A61L 2300/43; A61L 2300/64; A61L 2400/08; A61L 2430/00

(57) ABSTRACT

The present invention is directed to systems and/or biopolymer devices and methods of using the same. The systems and/or biopolymer devices described herein can be used for delivery of a fluid and/or molecules such as therapeutic agent(s), and/or for recruitment or collection of body fluids, e.g., for medical diagnosis. Methods of making and using the systems and/or biopolymer devices are also provided herein. In some embodiments, the systems and/or biopolymer devices can be used for continuous delivery of a therapeutic agent to repair or regenerate a tissue in a subject. In some embodiments, the biopolymer devices can be adapted for use as a catheter for fluid delivery to a target site.

### GOVERNMENT SUPPORT

This invention was made with government support under grant no. P41 EB002520 awarded by the National Institutes of Health (NIH); and grant no. W81XWH-08-2-0032 awarded by the US Army. The government has certain rights in the invention.

### TECHNICAL FIELD

The present disclosure relates generally to silk fibroin-based devices and methods of using the same. The silk fibroin-based devices can be used for delivery of fluids and/or molecules, such as therapeutic agent(s). In one aspect, methods for repairing or regenerating a tissue are also provided herein.

### BACKGROUND

Medical catheters have been employed in transferring medication fluids to local organs of a patient. See, e.g., Gandras E J. Minim Invasiv Ther 2009; 18(2):93-97; Niederlag W et al. Z Klin Med 1987; 42(20):1775-1777; and Kosinski D. et al., Postgrad Med 1998; 103(1):103-106, 109-110. Such catheters can be inserted into the patient's tissues for continuous local medication delivery or for the recruitment or collection of body fluids related to medical diagnosis. The catheters are generally formed of flexible and non-absorbable polymeric materials such as natural rubber latex (Nacey J N et a., Brit J Urol 1985; 57(3):325-328), polyvinyl chloride(Granados D L et al., J Biomed Mater Res 2001; 58(5):505-510), polyurethane (Izci Y et al., Neurol Res 2009; 31(3):234-237), polytetrafluoroethylene (Polner K et al. J Am Soc Nephrol 1995; 6(3):499-499), and silicon rubber (Nacey J N et a., Brit J Urol 1985; 57(3):325-328).

For medical applications, a catheter is generally in contact with living tissues for a period of time and should be biocompatible to avoid indwelling problems such as catheter-associated infections, encrustation and/or blockage. See, e.g., Eldin C et al. Med Mycol 2012; 50(6):627-630. In order to address this issue, for example, biodegradable materials, such as polyglycolides, polylactides, polybutylates, collagen, gelatin and hydrogels, can be integrated into catheter tubes or coated onto the inside lumen and/or outside wall of the catheter tubes. See, e.g., U.S. Pat. No. 5,201,724. However, the biodegradable materials can detach from the catheter surface over time. Furthermore, the remaining nonbiodegradable portions of the catheters generally need to be removed after certain periods of time. The removal process can disrupt the surrounding tissue, leading to additional complications, including, e.g., discomfort or pain during the removal process (Gwon H C et al. Journal of interventional cardiology 2006; 19(2):141-147) or localized infection. In cases where the catheter is placed in tissues with a complex configuration or structure, removal of the catheter can be challenging and increase the risk of tissue damage.

New therapeutic options to overcome at least some of the aforementioned limitations can be beneficial to clinical use. For example, a synthetic biodegradable epidural catheter composed of polylactide-co-glycolide has been previously discussed for use in procedures to administer anesthetic agents into the epidural space. See U.S. Pat. No. 5,201,724. Accordingly, there is still a need for mechanically robust, biodegradable, flexible, and biocompatible biomedical devices such as catheters, which can be inserted in tissues for continuous delivery of therapeutic agent(s) and/or for recruitment or collection of body fluids related to medical diagnosis, without complications and/or risks associated with subsequent removal of the biomedical devices.

### SUMMARY

Embodiments of various aspects described herein generally relate to systems, biopolymer devices and methods of making and using the same. For example, the systems and/or biopolymer devices described herein can be used for delivery of a fluid and/or agent(s) to a target site. In some embodiments, the target site can be an ex vivo tissue. In some embodiments, the target site can be an in vivo tissue or a tissue present in a subject. In some embodiments, the target site can be an anatomical repair site, e.g., a tissue to be treated, repaired or regenerated.

In one aspect, fluid delivery systems each comprising a perfusion device and a silk fibroin-based hollow tube are provided herein. The silk fibroin-based hollow tube extends along a longitudinal axis from a first end to a second end. The first end is adaptably connected to the perfusion device. The perfusion device can be an extracorporeal device or an implantable device. An exemplary perfusion device can comprise a perfusion pump.

In some embodiments, the perfusion device can further comprise a chamber configured to store a fluid to be delivered by the perfusion device into the hollow tube. In some embodiments, the fluid can further comprise at least one or more agents. Non-limiting examples of the agents include cells; therapeutic agents; small organic or inorganic molecules; saccharides; oligosaccharides; polysaccharides; biological macromolecules, e.g., peptides, proteins, and peptide analogs and derivatives; peptidomimetics; antibodies and antigen binding fragments thereof; nucleic acids; nucleic acid analogs and derivatives; glycogens or other sugars; immunogens; antigens; an extract made from biological materials such as bacteria, plants, fungi, or animal cells; animal tissues; naturally occurring or synthetic compositions; and any combinations thereof. In some embodiments, the agent can comprise an adipogenic agent (e.g., but not limited to insulin and/or dexamethasone). In some embodiments, the agent can comprise an angiogenic agent.

In some embodiments, the first end can be adaptably connected to the perfusion device using any methods known in the art. In some embodiments, the first end of the hollow tube can be adaptably connected to the perfusion device using a sealant. In some embodiments, the hollow tube can have two ends of different dimensions for connection to the perfusion device.

In some embodiments of this aspect and other aspects described herein, at least a portion of the hollow tube wall can be adapted to be permeable to a fluid and/or an agent to be delivered to a target site. For example, in some embodiments, at least a portion of the wall forming the hollow tube can be porous.

In any aspects described herein, the second end of the hollow tube can be sealed or opened. In some embodiments where the second end is sealed, silk fibroin can be used to seal the second end.

The fluid delivery systems described herein can be used in vivo or ex vivo. In some embodiments, the fluid delivery systems can be used to perfuse a tissue ex vivo. Accordingly, in another aspect, ex vivo systems for fluid delivery are also provided herein. The ex vivo system comprises a silk fibroin-based hollow tube and a tissue comprising at least a portion of the hollow tube disposed herein. The hollow tube extends along a longitudinal axis from a first end to a second end and at least one end is adapted to be capable of connecting to a connector and/or a device. The tissue to be perfused ex vivo can be derived from a subject or a tissue scaffold comprising cells.

In some embodiments, the ex vivo system can further comprise at least one connector and/or device adaptably connected to at least one end of the silk fibroin-based hollow tube. Exemplary connectors and/or devices include, but are not limited to, a fluid delivery tubing, a cannula, an adaptor, a perfusion device as described herein, or any combinations thereof. In some embodiments, the connector and/or device can be adapted for connection outside of the tissue. In other embodiments, the connector and/or device can be adapted for implantation in the tissue.

In some embodiments, the first end can be adaptably connected to at least one connector and/or device using any methods known in the art. In some embodiments, the first end of the hollow tube can be adaptably connected to the connector(s) and/or devices(s) using a sealant. In some embodiments, the hollow tube can have two ends of different dimensions for connection to the connector(s) and/or device(s).

A further aspect provides biopolymer devices comprising a silk fibroin-based hollow tube with at least one end adaptably connected to a connector and/or a device, and/or with two ends having different inside diameters. In some embodiments, the two ends of the silk fibroin-based hollow tube can differ in inside diameters by at least about 5% or more. Examples of the connector and/or device include, but are not limited to a fluid delivery tubing, a cannula, an adaptor, a perfusion device described herein, a pump, and any combinations thereof.

In some embodiments where the biopolymer device is adapted to form a catheter, the implanted biopolymer device do not necessarily require subsequent surgical removal upon tissue repair or regeneration, as the biopolymer device can degrade over time. For example, the biopolymer devices forming a catheter can be tuned to degrade as a tissue surrounding the devices gradually regenerates. Accordingly, in some embodiments, the biopolymer devices forming a catheter described herein can be designed as porous conduits to permit a passage or a uniform passage of a fluid (optionally comprising a therapeutic agent) across the hollow tube surface into the tissues to be treated, repaired or regenerated, while maintaining flexibility with sufficient strength to be stable within the anatomical region during surgery and/or into the postoperative period, with gradual absorption in the regenerating tissue.

In various aspects described herein, the silk fibroin-based hollow tube provides a conduit for continuous or intermittent fluid delivery and/or drug delivery directly to a target site. The silk-based hollow tube can comprise a nanopore or micropore wall structure. For example, in some embodiments, the porosity of the hollow tube can be adapted to mimic the porous wall of a blood vessel. Once implanted, at least a portion of the silk fibroin-based hollow tubes can be incorporated into the surrounding tissue for a period of time, for example, depending on degradation parameters of the device and the silk processing conditions used.

Methods for using one or more embodiments of the systems and/or biopolymer devices are also described herein. The method generally comprises placing at a target site at least a portion of the hollow tube of the system described herein, or of the ex vivo system described herein, or of the biopolymer device described herein.

The target site can be any volume or area of a material in need of perfusion. In some embodiments, the target site can be a tissue to be perfused with a fluid delivered by the system, the ex vivo system or the biopolymer device. In some embodiments, the target site can be a tissue to be repaired or regenerated. The tissue to be perfused can be ex vivo or present in a subject.

In some embodiments, the method can further comprise directing a fluid into the hollow tube, wherein the fluid optionally comprises an agent. In some embodiments, the fluid can permeate across at least a portion of the wall of the hollow tube to the target site over a period of time.

In some embodiments, the method can further comprise introducing cells at the target site. The introduced cells can surround at least a portion of the hollow tube. In some embodiments, at least 30% or more of the introduced cell volume can be retained at the target site over the period of time. In some embodiments, the method can further comprise growing cells surrounding the hollow tube. In some embodiments, the hollow tube can degrade as a tissue surrounding the target site remodels or regenerates.

In some embodiments, the methods described herein can be used for repair or regeneration of a tissue. Accordingly, yet another aspect provides methods for repairing or regenerating a tissue. The method comprises implanting in a tissue to be repaired or regenerated at least a portion of the hollow tube of the system described herein, or of the ex vivo system described herein, or of the biopolymer device, wherein the tissue is perfused with a fluid delivered by the system, ex vivo system or biopolymer device, thereby promoting repair or regeneration of the tissue. The tissue can be ex vivo or present in the subject. In some embodiments, the tissue to be repaired or regenerated can be a soft tissue.

In some embodiments, the method can further comprise further introducing cells into the tissue. Depending on the type of a tissue to be treated, in some embodiments, the cells can be derived from lipoaspirate. The cells can be introduced into the tissue by any methods known in the art. For example, the cells can be delivered via the system, ex vivo system or biopolymer device described herein, and/or implanted in the tissue prior to, concurrently with, or after the implantation of the hollow tube.

In some embodiments, the method can further comprise introducing the fluid into the hollow tube. In some embodiments, the fluid can permeates across at least a portion of a wall forming the hollow tube.

In some embodiments of this aspect and other aspects described herein, the fluid can comprise an agent. The agent can be any molecule or entity that facilitates repair and/or regeneration of a tissue, and vary with types of a tissue to be treated. Non-limiting examples of such agents include cells (including, e.g., stem cells); therapeutic agents; small organic or inorganic molecules; saccharides; oligosaccharides; polysaccharides; biological macromolecules, e.g., peptides, proteins, and peptide analogs and derivatives; peptidomimetics; antibodies and antigen binding fragments thereof; nucleic acids; nucleic acid analogs and derivatives; glycogens or other sugars; immunogens; antigens; an extract made from biological materials such as bacteria, plants, fungi, or animal cells; animal tissues; naturally occurring or synthetic compositions; and any combinations thereof. In some embodiments, such agent can comprise a therapeutic agent. In some embodiments, such agent can comprise an adipogenic agent (e.g., but not limited to insulin and/or dexamethasone). In some embodiments, such agent can comprise a pro-angiogenic agent.

Other features and advantages of the invention will be apparent from the detailed description, and from the claims. Thus, other aspects of the invention are described in the following disclosure and are within the ambit of the invention.

### BRIEF DESCRIPTION OF THE DRAWINGS

FIGS. 1A-1D is a set of SEM images of silk tubes produced using multiple dip-coating method in accordance with one or more embodiments described herein. FIG. 1A shows a fractured cross-section of a silk tube formed by dip-coating a wire in a silk fibroin solution for 3 times. FIGS. 1B-1D show a fractured cross-section, a magnified fractured cross-section and side view of a silk tube formed by dip-coating a wire in a silk fibroin solution for 5 times, respectively.

FIGS. 2A-2C is a set of SEM images of silk tubes produced using gel spinning in accordance with one or more embodiments described herein. FIG. 2A shows a fractured cross-section of the silk tube. FIG. 2B shows a magnified fractured cross-section of the silk tube. FIG. 2C shows the side view of the silk tube.

FIGS. 3A-3C show design, fabrication and characterization of silk tubes in accordance with one or more embodiments described herein. FIG. 3A is a set of pictures showing the fabrication of gel spun silk tubes. In the left image, a polypropylene tube (˜1090 μm outer diameter) was assembled to make one end of a resultant silk tube with a larger inner lumen fitting. In the middle image, highly concentrated silk solution was spun on the rotating metal wire. In the right image, the gel spun silk tube was obtained after freeze-drying. FIG. 3B is a schematic of one embodiment of a silk fibroin-based catheter described herein. The other end of tube was capped with silk by dipping one end into concentrated silk solution and drying. FIG. 3C is a set of pictures showing that the gel spun tubes are flexible even when dried.

FIG. 4 is a plot of tensile stress against tensile strain of the silk tubes measured under physiological conditions-hydrated in saline solution at 37° C.

FIG. 5 is a graph of cumulative insulin concentration over time based on release of insulin from a silk fibroin-based catheter over a period of time. Study was conducted using an osmotic pump attached to a silk fibroin-based catheter immersed in PBS solution at 37° C.

FIGS. 6A-6B is a set of pictures showing fluid flow across the silk fibroin-based catheter wall along the length of the silk fibroin-based catheter, as visualized by a blue dye in PBS. FIG. 6A shows that a silk fibroin-based catheter was inserted into a blunt-tipped needle, and a sealant was applied at the free end and at the junction between the needle and the catheter (arrows). FIG. 6B is a set of pictures showing that FD&C Blue 1 dye solution was slowly infused into a PBS bath from the porous wall of the silk fibroin-based catheter along its length, and did not appear to leak from the ends where the sealant was applied.

FIGS. 7A-7B are data graphs showing in vitro continuous release of drugs from the porous silk fibroin-based catheters over time in a controlled fashion. The silk fibroin-based catheters were attached to an osmotic pump delivering the drugs at a flow rate of ˜0.5 μL/hr. FIG. 7A shows a cumulative dexamethasone concentration in PBS solution over time. FIG. 7B shows a cumulative insulin concentration in PBS solution over time.

FIGS. 8A-8B shows an animal model of perfusion drug delivery. FIG. 8A is a picture showing one embodiment of a silk fibroin-based catheter attached to an implantable mini-osmotic pump. FIG. 8B is a hematoxylin and eosin (H&E) image of a silk fibroin-based catheter within a fat graft 28 days post-implantation.

FIGS. 9A-9B show experimental data of dexamethasone dose response in volume retention of fat grafts 28 days post-implantation. FIG. 9A is a bar graph showing volume of fat grafts post-implantation with or without a silk fibroin-based catheter that was connected to an implantable osmotic pump (ALZET, Durect Corp.) that continuously released different concentrations of dexamethasone at a rate of about 0.11 μL/hr. The volumes of the harvested fat grafts were measured using gas pycnometry (AccuPyc II). The dashed lines indicate the original implanted volume of 0.5 ml of lipoaspirate. There appeared to be no statistically significant differences detected between groups; however the largest effect was detected with the ˜2 mg/ml dose of dexamethasone. The grafts with no tissue fiber did not show a significant effect between the groups, which indicate that the lipoaspirate-only controls were not affected by the release of dexamethasone into the fat graft containing the silk fibroin-based catheter. FIG. 9B is a set of hematoxylin and eosin (H&E) images of silk fibroin-based catheters releasing dexamethasone within fat grafts 28 days post-implantation. There was little to no cellular response to the silk fibroin-based catheter placement (outlined with dashed circles when appropriate) with the vehicle control, and the ˜1 and ˜2 mg/ml doses of dexamethasone. In some embodiments, there could be a mild inflammatory response in the area immediately surrounding the tissue fiber at the ˜3 mg/ml dose of dexamethasone. Thus, these results shows the feasibility of using a silk fibroin-based catheter as a conduit for localized drug delivery.

FIGS. 10A-10B show experimental data of insulin dose response in volume retention of fat grafts 28 days post-implantation. FIG. 10A is a bar graph showing volume of fat grafts post-implantation with or without a silk fibroin-based catheter that was connected to an implantable osmotic pump (ALZET, Durect Corp.) that continuously released different concentrations of insulin at a rate of about 0.11 μL/hr. The volumes of the harvested fat grafts were measured using gas pycnometry. The dashed lines indicate the original implanted volume of 0.5 ml of lipospirate. Although there appeared to be no statistical significance, the group with the highest volume retention was ˜300 μg of insulin. FIG. 10B is a set of hematoxylin and eosin (H&E) images of silk fibroin-based catheters releasing insulin within fat grafts 28 days post-implantation. There was little to no cellular response to the silk fibroin-based catheter with the insulin doses and vehicle control. However, a fibrous tissue was observed to surround the silk fibroin-based catheter with a ˜1000 μg dose of insulin. Thus, a porous hollow silk fibroin-based catheter can be used as a conduit for drug delivery to infuse therapeutic agents such as adipogenic agents (e.g., insulin) into tissues, e.g., soft tissue grafts.

### DETAILED DESCRIPTION OF THE INVENTIONS

The present disclosure provides a solution to at least one of the problems associated with implantation of catheters in a tissue for a period of time. The systems and/or biopolymer devices described herein (including, but are not limited to silk fibroin-based catheters) were developed to reduce or prevent indwelling problems such as catheter-associated infections, encrustation and/or blockage, as well as to minimize or eliminate risk of damaging the treated or regenerated tissue during subsequent removal of the implanted systems and/or devices. In solving these problems, the inventors have, inter alia, developed silk fibroin-based porous and biodegradable catheters for continuous or intermittent release of a fluid and/or a therapeutic agent to a target site. Specifically, as proof of principle, the inventors have implanted the silk fibroin-based catheters, which are applicable to any target site ex vivo or in vivo for any period of time, in a tissue in an animal model, e.g., for about 1 month, demonstrating the silk fibroin-based catheters can be implanted in tissues to deliver a fluid and/or agent that can promote tissue repair and/or regeneration. In some time, the inventors have showed that the silk fibroin-based catheters releasing an adiopogenic agent can promote repair and/or regeneration of an adipose tissue.

### Systems and Biopolymer Devices Comprising a Silk Fibroin-Based Hollow Tube

In one aspect, provided herein relates to systems, e.g., for delivery of a fluid and/or agent to a target site. The target site can be ex vivo or in vivo. In some embodiments, the system comprises (a) a perfusion device; and (b) a hollow tube extending along a longitudinal axis from a first end to a second end; wherein the hollow tube is formed of a material comprising silk fibroin, and wherein the first end is adaptably connected to the perfusion device.

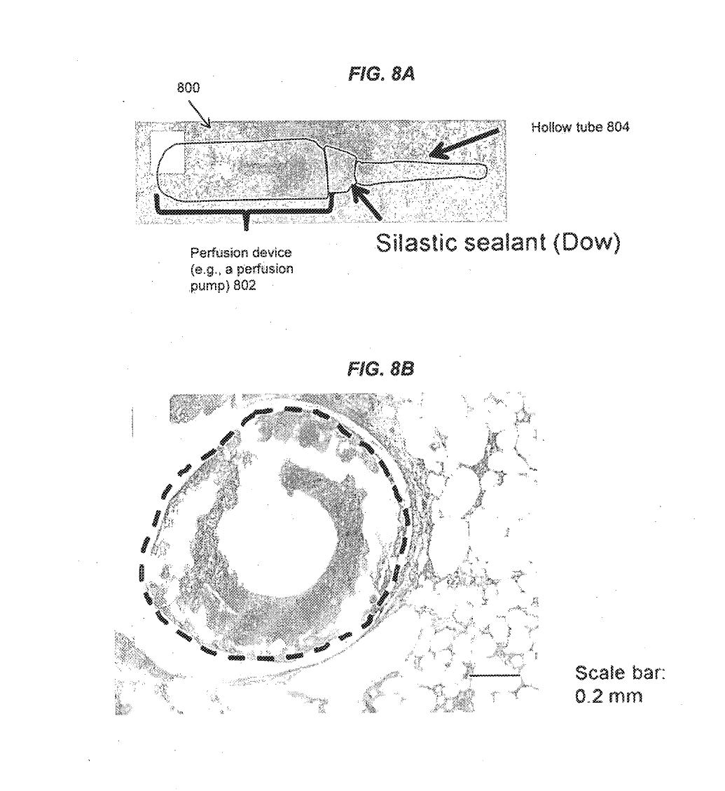

FIG. 8A is a picture of a system according to one embodiment described herein. The comprises a perfusion device 802 and a hollow tube 804 with one end adaptably connected to the perfusion device 802. The end of the hollow tube can be adaptably connected to the perfusion device using any methods known in the art. For example, a sealant can be used to connect one end of the hollow tube to the perfusion device. In some embodiments, the end of the hollow tube adaptably connected to the perfusion device can have an inside diameter configured to permit coupling of the hollow tube to the perfusion device. Accordingly, in some embodiments, both ends of the hollow tube can different inside diameters.

In some embodiments, the hollow tube can be adapted to permit passage of a fluid to be released across at least a portion of a wall forming the hollow tube. In some embodiments, the at least a portion of the wall forming the hollow tube is porous. In some embodiments, the end that is not connected to a perfusion device can be left open or sealed. In one embodiment, the end that is not connected to a perfusion device can be sealed such that a fluid permeates through a porous wall of the hollow tube to reach a target site or area. In some embodiments, the end that is not connected to a perfusion device can be sealed with silk fibroin. Additional description about the hollow tubes can be found below and is applicable for use in the systems described herein.

As used herein, the term “perfusion device” refers to a device or an element designed to facilitate delivery of or flow a fluid, which optionally comprises an agent, to a target site or through a target area. The target site or target area can be present ex vivo (i.e., outside the body of a subject) or in vivo (i.e., inside the body of a subject). Accordingly, in some embodiments, the target site or target area can be a tissue, an organ, an organoid (e.g., a three-dimensional structure of cells that has some of the features of an organ), a tissue scaffold comprising cells, or any combinations thereof. In these embodiments, the design of the perfusion device can be configured to suit the need of various ex vivo or in vivo applications. Examples of a perfusion device include, but are not limited to, a pump (including, but not limited to, a perfusion pump, an osmotic pump, a centrifugal pump, an axial-flow roller pump), a flow sensor, a pressure sensor, a temperature sensor, a level sensor, an air embolus sensor, an occlude, a valve, a flow splitter, or any combinations thereof.

In some embodiments, the perfusion device can be an extracorporeal device (e.g., a device that is used outside of the body). In these embodiments, at least a portion of the hollow tube can be implanted at a target site or area, while the perfusion device adaptably connected to the hollow tube can remain outside a target site or area. For example, the perfusion device can be operated outside a tissue, organ, organoid, or a tissue scaffold comprising cells, while at least a portion of the hollow tube is implanted therein. In some embodiments, the perfusion device can be operated outside the body of a subject while connecting to the hollow tube implanted in a tissue or tissue scaffold at a target site.

In some embodiments, the perfusion device can be an implantable device. In these embodiments, the entire system can be implanted at a target site or area. In some embodiments, the entire system can be implanted in a tissue, organ, organoid, or a tissue scaffold comprising cells. In some embodiments, the entire system can be implanted in a tissue or tissue scaffold comprising cells present in a subject.

The perfusion device can be configured to flow a fluid (optionally comprising at least one agent) at a selected flow rate. The flow rate of the fluid can range from nanoliter(s) per hour to milliter(s) per hour. In some embodiments, the perfusion device can permit a flow of fluid at a rate such that a desired therapeutic outcome is obtained at a target site or area. In some embodiments, the perfusion device can permit a flow of fluid at a rate such that an effective amount of an agent, if any, is delivered to a target site or area. As used herein, the term “effective amount” refers to an amount that is sufficient to induce or yield a desired outcome or a desired therapeutic outcome. In some embodiments, the effective amount refers to an amount of an agent that is sufficient to promote tissue repair or regeneration. Accordingly, the perfusion device can be configured to deliver a fluid to a target site or area, based on, e.g., desirable drug release, size of target volume/area to be perfused, types of pumps, dimensions and/or design of the hollow tube to which the perfusion is connected to, or any combinations thereof.

In some embodiments, the perfusion device can be configured to flow a fluid at a rate of about 50 nL/hr to about 5 mL/hr or about 75 nL/hr to about 3 mL/hr or about 100 nL/hr to about 1 mL/hr. In some embodiments, the perfusion device can permit a flow of fluid at a rate of at least about 50 nL/hr, at least about 100 nL/hr, at least about 250 nL/hr, at least about 500 nL/hr, at least about 750 nL/hr, at least about 1000 nL/hr, at least about 5 μL/hr, at least about 10 μL/hr, at least about 50 μL/hr, at least about 100 μL/hr, at least about 250 μL/hr, at least about 500 μL/hr, at least about 750 μL/hr, at least about 1000 μL/hr, at least about 2 mL/hr, at least about 3 mL/hr, at least about 4 mL/hr, at least about 5 mL/hr. In some embodiments, the perfusion device can permit a flow of fluid at a rate of no more than 50 nL/hr, no more than 100 nL/hr, no more than 250 nL/hr, no more than 500 nL/hr, no more than 750 nL/hr, no more than 1000 nL/hr, no more than 5 μL/hr, no more than 10 μL/hr, no more than 50 μL/hr, no more than 100 μL/hr, no more than 250 μL/hr, no more than 500 μL/hr, no more than 750 μL/hr, no more than 1000 μL/hr, no more than 2 mL/hr, no more than 3 mL/hr, no more than 4 mL/hr, no more than 5 mL/hr.

In some embodiments, the perfusion device can further comprise at least one or more (e.g., 1, 2, 3 or more) chambers configured to store one or more fluids to be delivered by the perfusion device into the hollow tube. In some embodiments, the fluid can further comprise at least one or more (e.g., 1, 2, 3, 4, 5 or more) agents. An agent can be any desirable molecule or entity to be introduced into a target site or area. Examples of an agent include, without limitations, cells; therapeutic agents; small organic or inorganic molecules; saccharides; oligosaccharides; polysaccharides; biological macromolecules, e.g., peptides, proteins, and peptide analogs and derivatives; peptidomimetics; antibodies and antigen binding fragments thereof; nucleic acids; nucleic acid analogs and derivatives; glycogens or other sugars; immunogens; antigens; an extract made from biological materials such as bacteria, plants, fungi, or animal cells; animal tissues; naturally occurring or synthetic compositions; and any combinations thereof.

In some embodiments, the agent can comprise an adipogenic agent. Examples of adipogenic agents include, but are not limited to, insulin, dexamethasone, IGF-1, cAMP, glucocorticoid, triiodothyronine, indomethacin, IBMX, thiazolidinediones, glitazones, rosiglitazone and similar agents, mammalian adipogenic factors described in the International Patent Appl. No. WO 1991/018924, and any combinations thereof. The content of the patent application is incorporated herein by reference. In some embodiments, the adipogenic agent stored in the perfusion device can comprise insulin and/or dexamethasone. In some embodiments, the agent can comprise a pro-angiogenic agent as defined below.

In some embodiments, for example, as shown in FIG. 8A, one end of the hollow tube 804 can be adaptably connected to a perfusion device, e.g., a perfusion pump 802.

In some embodiments, the system described herein can be used ex vivo. For example, at least a portion of the hollow tube can be implanted into a tissue, organ, organoid, or a tissue scaffold that are cultured ex vivo, while the perfusion device can be externally operated to perfuse the sample. Accordingly, ex vivo systems, e.g., for fluid delivery, are also provided herein. The ex vivo system comprises (a) a hollow tube extending along a longitudinal axis from a first end to a second end; wherein the hollow tube is formed of a material comprising silk fibroin, and at least one end is adapted to be capable of connecting to a connector and/or a device; and (b) a tissue comprising at least a portion of the hollow tube disposed therein. In some embodiments, the tissue can be collected or derived from a subject. In some embodiments, the tissue can be a tissue scaffold comprising cells. In some embodiments, the tissue can be an organoid.

In some embodiments, the ex vivo system can further comprise a connector and/or device adaptably connected to at least one end of the hollow tube. Examples of a connector and/or device include, but are not limited to, a fluid delivery tubing, a cannula, an adaptor, a perfusion device as described herein, or any combinations thereof. In some embodiments, the connector and/or device can be adapted for connection outside of the tissue. In some embodiments, the connector and/or device can be adapted for implantation in the tissue.

Different features of the systems and hollow tubes as described herein and throughout the specification can be applied to the ex vivo systems alone or in any combinations.

In a further aspect, provided herein relates to biopolymer devices, e.g., for delivery of a fluid and/or agent to a target site. In one embodiment, the target site can be a tissue to be treated, repaired or regenerated in a subject. In another embodiment, the target site can be a tissue cultured ex vivo. In some embodiments, the biopolymer devices can comprise a hollow tube extending along a longitudinal axis from a first end to a second end, wherein the hollow tube is formed of a material comprising silk fibroin; and wherein the first end is further adaptably connected to a connector and/or an adaptor. In these embodiments, the connector and/or adaptor can include a fluid delivery tubing, cannula, and/or an implantable perfusion pump. In some embodiments, the fluid delivery tubing or cannula can be made of a material comprising polypropylene.

In some embodiments, the biopolymer device comprising a hollow tube can be adapted to form a catheter. As used herein, the term “catheter” generally refers to an elongated tubular structure, of which a least a portion can be inserted into the body (e.g., a body cavity, duct, vessel, a tissue void) of a subject, e.g., to permit introduction or withdrawal of a fluid, e.g., liquids and/or air, to keep a passageway (e.g., urethra) open, to treat diseases or disorder, and/or to perform a surgical procedure. By modifying the material, design and/or manufacturing process of catheters, catheters can be tailored for various applications, e.g., but not limited to, cardiovascular, urological, gastrointestinal, neurovascular, ophthalmic, tissue repair or regeneration applications. Catheters can be left inside the body, either temporarily or permanently, and referred to as an indwelling catheter.

In some embodiments, the biopolymer device comprising a hollow tube can be adapted to form a stent. As used herein, the term “stent” generally refers to a scaffolding device or structure that provides support to a surrounding tissue and/or maintains the patency and/or integrity of a body passageway. In some embodiments, the stent can have a tubular body structure. In some embodiments, the stent can be used to maintain the patency and/or integrity of a blood vessel.

Hollow tube: The hollow tube extends along a longitudinal axis from a first end to a second end. In some embodiments, the inside diameter of the hollow tube at the first end being different from the inside diameter of the hollow tube at the second end. In some embodiments, the inside diameter of the hollow tube at the first end is substantially different from the inside diameter of the hollow tube at the second end. The hollow tube is generally formed of a material comprising silk fibroin.

The outside diameter of the hollow tube at the two ends can have the same or different dimensions, regardless of the respective inside diameters of the hollow tube at the two ends. For example, the wall thickness of the hollow tube at the two ends can vary to yield the outside diameters of the same or different dimensions at the two ends. By way of example only, the wall thickness can differ at the two ends such that the outside diameters at the two ends are different while the inside diameters at the two ends are substantially the same. In another instance, the wall thickness can differ at the two ends such that the outside diameter at the two ends are substantially the same while the inside diameters at the two ends are different.

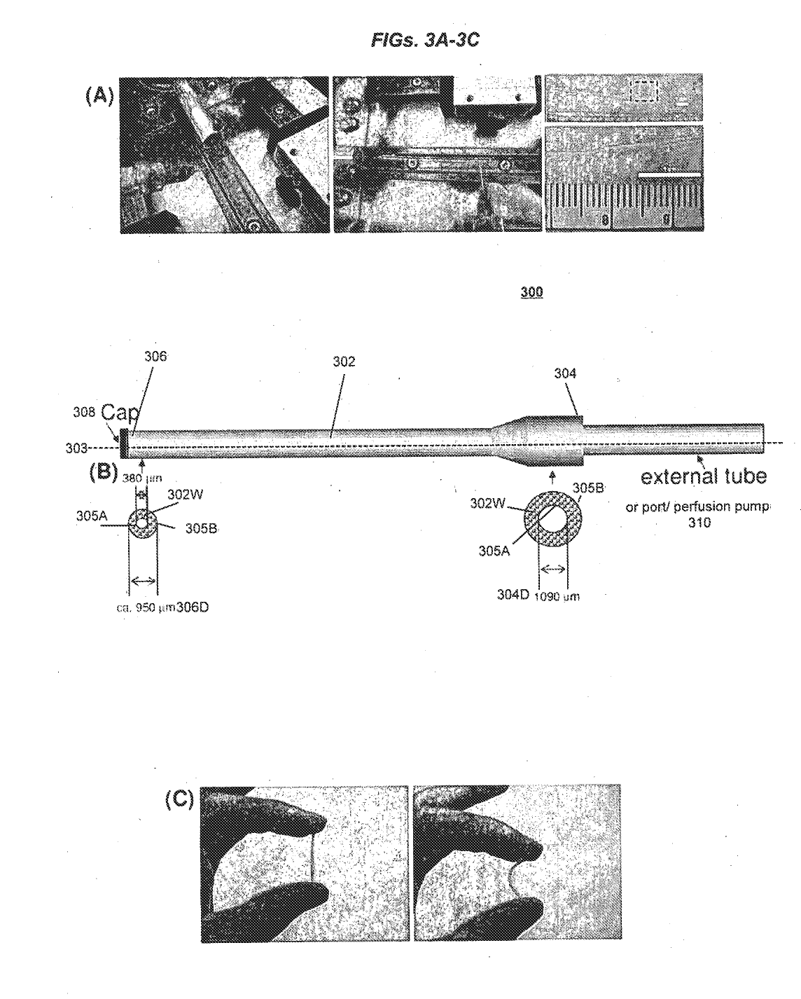

FIG. 3B shows a diagrammatic view of a biopolymer device according to one embodiment described herein. The biopolymer device 300 comprises a hollow tube 302 extending along a longitudinal axis 303 from a first end 304 to a second end 306. The inside diameter 304D of the hollow tube at the first end is larger than the inside diameter 306D of the hollow tube at the second end.

While FIG. 3B shows a hollow tube having a circular lumen cross-section, the lumen cross-section of the hollow tube can adopt any other shape. For example, the lumen cross-section of the hollow tube can be square, rectangular, triangular, oval, elliptical, diamond-shaped, polygonal-shaped, irregular-shaped, or any combinations thereof. Accordingly, the term “inside diameter” as used herein generally refers to a characteristic width of the lumen of the hollow tube in any shape, and is not construed to be limiting the shape of the lumen to be circular.

In some embodiments, the inside diameter of the hollow tube can vary linearly or non-linearly along the longitudinal axis 303 from the first end 304 to the second end 306. In some embodiments, the inside diameter of the hollow tube can remain constant along the longitudinal axis 303 for the majority portion of the tube length except in the portion(s) approaching to the first end 304 and/or the second end 306. For example, FIG. 3B shows that the majority portion of the hollow tube along the longitudinal axis 303 can have an inside diameter substantially the same as the inside diameter at the second end. When approaching toward the first end, the inside diameter of the hollow tube can then increase to a desired size, e.g., sufficient to permit attachment to or fitting with a connector, an adaptor and/or a fluid delivery device.

In some embodiments, the inside diameter 304D of the hollow tube at the first end can be substantially larger than the inside diameter 306D of the hollow tube at the second end. For example, the inside diameter 304D of the hollow tube at the first end can be larger than the inside diameter 306D of the hollow tube at the second end by at least about 10%, at least about 20%, at least about 30%, at least about 40%, at least about 50%, at least about 60%, at least about 70%, at least about 80%, at least about 90%, at least about 95% or more. In some embodiments, the inside diameter 304D of the hollow tube at the first end can be larger than the inside diameter 306D of the hollow tube at the second end by at least about 1.1-fold, at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold or more.

The inside diameter 304D of the hollow tube at the first end can be generally of any size. In some embodiments, the inside diameter 304D of the hollow tube at the first end can range from about 300 μm to about 10 mm, about 400 μm to about 5 mm, about 500 μm to about 3 mm, or about 750 μm to about 2 mm. In some embodiments, the inside diameter 304D of the hollow tube at the first end can be about 500 μm to about 1500 μm. In some embodiments, the inside diameter 304D of the hollow tube at the first end can be about 750 μm to about 1250 μm.

In some embodiments, the inside diameter 304D of the hollow tube at the first end can be adapted to connect to a connector, an adaptor, and/or a fluid delivery device 310. Examples of the connector, adaptor and/or fluid delivery device include, but are not limited to, fluid delivery tubings, cannulas, ports, valves, perfusion devices, and/or pumps. Depending on the desirable flow rate of a fluid and/or drug release, body size of a subject, types of the pumps, and/or the size of the biopolymer device, the pump can be configured to provide a flow rate ranging from nanoliters per hour to milliliters per hour. In some embodiments, the inside diameter 304D of the hollow tube at the first end can be adapted to connect to an implantable pump, including, e.g., but not limited to an osmotic pump and/or perfusion pump. In these embodiments, the implantable pump can be configured to provide a flow rate of about 50 nL/hr to about 1000 μL/hr or about 75 nL/hr to about 750 μL/hr or about 100 nL/hr to about 500 μL/hr.

Accordingly, in some embodiments, the biopolymer device 300 can further comprise a connector, adaptor and/or fluid delivery device 310 adaptably connected to the first end 304 of the hollow tube. In some embodiments, the biopolymer device 300 can further comprise a connector and/or adaptor (e.g., a fluid delivery tubing or tube) 310 adaptably connected to the first end 304 of the hollow tube. In one embodiment, the biopolymer device 300 can further comprise a polypropylene tube or cannula adaptably connected to the first end 304 of the hollow tube. In some embodiments, the biopolymer device 300 can further comprise an implantable perfusion device (e.g., but not limited to an osmotic pump and/or a perfusion pump) adaptably connected to the first end 304 of the hollow tube.

In some embodiments, the implantable perfusion device can store a therapeutic agent to be infused to a target tissue. Examples of a therapeutic agent for perfusion include, without limitations, cells; therapeutic agents; small organic or inorganic molecules; saccharides; oligosaccharides; polysaccharides; biological macromolecules, e.g., peptides, proteins, and peptide analogs and derivatives; peptidomimetics; antibodies and antigen binding fragments thereof; nucleic acids; nucleic acid analogs and derivatives; glycogens or other sugars; immunogens; antigens; an extract made from biological materials such as bacteria, plants, fungi, or animal cells; animal tissues; naturally occurring or synthetic compositions; and any combinations thereof. In some embodiments, the therapeutic agent can comprise an adipogenic agent as described below. Exemplary adipogenic agents include, without limitations, insulin and/or dexamethasone. In some embodiments, the therapeutic agent can comprise an angiogenic agent as described below.

In some embodiments, the inside diameter 306D of the hollow tube at the second end can be generally of any dimension. In some embodiments, the inside diameter 306D of the hollow tube at the second end can range from about 50 μm to about 2000 μm, about 100 μm to about 1500 μm, about 150 μm to about 1000 μm, or about 300 μm to about 750 μm. In some embodiments, the inside diameter 306D of the hollow tube at the second end can be about 250 μm to about 500 μm. In some embodiments, the inside diameter 306D of the hollow tube at the second end can be about 300 μm to about 400 μm.

In some embodiments, the second end 306 of the hollow tube can be at least partially open or fully open. The term “open” as used herein generally refers to an end of the hollow tube which allows for the passage of fluid through the end. Accordingly, in some embodiments, the second end 306 of the hollow tube can permit passage of at least 10% or more of a fluid entering from the first end therethrough. In some embodiments, the second end 306 of the hollow tube can permit passage of more than 10%, including, at least about 15%, at least about 20% at least about 30%, at least about 40%, at least about 50%, at least about 60%, at least about 70%, at least about 80%, at least about 90%, at least about 95%, at least about 98% or up to 100% of a fluid entering from the first end therethrough.

In some embodiments, the second end 306 of the hollow tube can be closed or sealed. The term “closed” or “sealed” as used herein refers to an end of the hollow tube which does not allow for the passage of fluid through the end. In these embodiments, the second end 306 of the hollow tube can be closed or sealed with a cap 308 or seal. The cap 308 or seal can be formed integral to the hollow tube during the fabrication process, or it can be independently fitted over or into the originally-open second end 306 of the hollow tube. The cap 308 or seal can be produced from any art-recognized biocompatible material(s). Examples of the biocompatible materials that can be used in the cap 308 or seal include, but are not limited to, silk or silk fibroin, poly-dimethyl-siloxane (PDMS), poly-glycerol-sebacate (PGS), polylactic acid (PLA), poly-L-lactic acid (PLLA), poly-D-lactic acid (PDLA), polyglycolide, polyglycolic acid (PGA), polylactide-co-glycolide (PLGA), polydioxanone, polygluconate, polylactic acid-polyethylene oxide copolymers, modified cellulose, collagen, polyhydroxybutyrate, polyhydroxpriopionic acid, polyphosphoester, poly(alpha-hydroxy acid), polycaprolactone, polycarbonates, polyamides, polyanhydrides, polyamino acids, polyorthoesters, polyacetals, polycyanoacrylates, degradable urethanes, aliphatic polyesterspolyacrylates, polymethacrylate, acyl substituted cellulose acetates, non-degradable polyurethanes, polystyrenes, polyvinyl chloride, polyvinyl flouride, polyvinyl imidazole, chlorosulphonated polyolifins, polyethylene oxide, polyvinyl alcohol, nylon silicon, poly(styrene-block-butadiene), polynorbornene, and hydrogels. Other suitable polymers can be obtained by reference to The Polymer Handbook, 3rd edition (Wiley, N.Y., 1989). Combinations of these polymers can also be used.

In some embodiments where the biopolymer device is desired to be biodegradable or resorbable such that no physical removal of any component is subsequently required, the cap 308 or seal can be produced from one or more biodegradable materials. Examples of biodegradable materials include, but are not limited to, silk or silk fibroin, polyester, polyamide, polycarbonate, polyhydroxyacids (e.g., polylactic acid, polyglycolic acid, polycaprolactone, and any combinations thereof. In one embodiment, the cap 308 or seal can comprise a silk fibroin matrix.

In some embodiments, while the cap 308 or seal at the second end 306 of the hollow tube does not allow for the passage of a bulk fluid through the second end, the cap 308 or seal can be made of a material that is permeable to a fluid such that the fluid can diffuse or permeate through the material of the cap or seal over a period of time. In one embodiment, the cap 308 or seal can comprise a porous silk fibroin matrix.

The hollow tube can be of any length, for example, depending on the size of a target site or tissue to be treated. In some embodiments, the hollow tube can have a length of at least about 3 mm or more. In some embodiments, the hollow tube can have a length of more than 3 mm, including, e.g., at least about 4 mm, at least about 5 mm, at least about 6 mm, at least about 7 mm, at least about 8 mm, at least about 9 mm, at least about 10 mm, at least about 11 mm, at least about 12 mm, at least about 13 mm, at least about 14 mm, at least about 14 mm, at least about 15 mm, at least about 20 mm, at least about 25 mm, at least about 30 mm, at least about 40 mm, at least about 50 mm, at least about 60 mm, at least about 70 mm, at least about 70 mm, at least about 80 mm, at least about 90 mm, at least about 100 mm or longer. The length of the hollow tube can vary with different applications and/or sites of implantation. Accordingly, in some embodiments, the length of the hollow tube can be smaller than 3 mm or longer than 100 mm. In some embodiments, the length of the hollow tube can range from about 1 cm to about 5 cm. In some embodiments, the length of the hollow tube can range from about 1 cm to about 3 cm.

The wall 302W forming the hollow tube can be non-porous (e.g., impermeable) or porous (e.g., permeable or selectively permeable). Accordingly, the wall 302W forming the hollow tube can have a porosity of about 0% to about 99%. As used herein, the term “porosity” is a measure of total void space (e.g., through-holes, openings, interstitial spaces, and/or hollow conduits) in a material, and is a fraction of volume of total voids over the total volume, as a percentage between 0 and 100% (or between 0 and 1). The hollow tube wall 302W with substantially zero porosity is non-porous or non-permeable. As used interchangeably herein, the terms “non-porous” and “non-permeable” refer to a material or matrix that does not allow any molecule or substance to pass through. Determination of matrix porosity is well known to a skilled artisan, e.g., using standardized techniques, such as mercury porosimetry and gas adsorption, e.g., nitrogen adsorption.

In accordance with some embodiments of the invention, the hollow tube wall 302W can be adapted to be porous and thus allow a fluid and/or one or more agents to diffuse or permeate through the tube wall 302W to a target site. Accordingly, the hollow tube wall 302W can be adapted to permit passage of at least one or more agents (e.g., at least two or more agents) to be released across at least a portion of the tubular wall 302W.

As used herein, the term “porous” generally refers to a material or matrix that is permeable or selectively permeable. The term “permeable” as used herein means a material or matrix that permits passage of a fluid (e.g., liquid or gas), and/or at least one agent, e.g., but not limited to, a molecule, and/or a whole living cell. The term “selectively permeable” as used herein refers to a material or matrix that permits passage of one or more target group or species, but acts as a barrier to non-target groups or species. For example, a selectively-permeable hollow tube wall 302W can allow passage of a fluid (e.g., liquid and/or gas) and/or a specific agent introduced into the lumen of the hollow tube from the inner wall surface 305A to the outer wall surface 305B, but does not allow for the passage of other undesirable agents through the wall. The permeability of the tubular wall 302W to individual matter/species can be determined based on a number of factors, including, e.g., material property of the hollow tube wall 302W (e.g., pore size, and/or porosity), interaction and/or affinity between the wall material and individual species/matter, individual species size, concentration gradient of individual species between both sides of the wall surfaces 305A and 305B, elasticity of individual species, and/or any combinations thereof.

In some embodiments, the porous tubular wall 302W can comprise through-holes or pore apertures extending vertically and/or laterally between the inner wall surface 305A and outer wall surface 305B. The through-hole or pore apertures can be formed, e.g., by laser cutting or laser etching. See, e.g., the international patent application no. PCT/US12/34401 filed Apr. 20, 2012, the content of which is incorporated herein by reference. In some embodiments, the porous tubular wall 302W can comprise a connected network of pores or void spaces (which can, for example, be openings, interstitial spaces or hollow conduits) throughout its volume. For example, FIGS. 2A-2C show that the tubular wall 302W comprises a connected network or pores or void spaces. In these embodiments, the porous tubular wall 302W can comprise micropores. By way of example only, the micropores can have a size ranging from about 1 μm to about 100 μm or from about 5 μm to about 75 μm or from about 10 μm to about 50 μm.

The wall thickness of the hollow tube can vary with a number of factors, including, e.g., but not limited to, fabrication methods, desired diffusion rate or release rate of an agent to be delivered across the tubular wall, mechanical properties of the hollow tube (e.g., strength and flexibility), degradation lifetime, and any combinations thereof. In some embodiments, the wall thickness of the hollow tube can range from about 15 μm to about 1500 μm, or from about 30 μm to about 1000 μm, or from about 50 μm to about 500 μm. In some embodiments, the wall thickness of the hollow tube can range from about 100 μm to about 150 μm. In some embodiments, the wall thickness of the hollow tube can range from about 150 μm to about 300 μm. In some embodiments, the wall thickness of the hollow tube can range from about 300 μm to about 400 μm.

The mechanical properties of the hollow tube can be tuned to suit the need of different applications. For example, flexibility of the hollow tube can be tuned to facilitate implantation of the biopolymer device into tortuous or irregular-shape anatomical implantation sites. Alternatively or additionally, stiffness of the hollow tube can be tuned to yield a desired degradation profile of the hollow tube and/or to mimic mechanical compliance of a tissue surrounding the implantation site. Accordingly, in some embodiments, the hollow tube, when hydrated, can have a tensile modulus or elastic modulus of about 0.1 MPa to about 15 MPa, or about 0.5 MPa to about 10 MPa, or about 1 MPa to about 8 MPa, or about 1.5 MPa to about 6 MPa, or about 1.5 MPa to about 4 MPa. In some embodiments, the hollow tube, when hydrated, can have a tensile modulus or elastic modulus comparable to (e.g., within 15% or less) that of a soft tissue surrounding the implantation site. For example, arteries and veins generally have an elastic modulus of ˜0.3-5 MPa, muscle ˜0.8 MPa, liver and kidney ˜10 MPa, and spinal cord ˜2 MPa. As used herein, the term “tensile modulus,” also known as “Young's modulus” or “elastic modulus,” is generally a measure of the stiffness of a material. The tensile modulus or elastic modulus of the hollow tube can be determined by any art-recognized method or the methods described in the Examples. For example, the hollow tubes can be hydrated, e.g., in a buffered solution such as PBS, to reach swelling equilibrium prior to mechanical analysis. The hydrated hollow tubes can then be subjected to tensile tests such as uniaxial tensile tests to generate stress/strain curves, from which the tensile moduli can be determined by computing the slope of the initial, linear portion of the stress-strain curves.

In some embodiments, the ultimate tensile strength of the hollow tube, when hydrated, can range from about 0.1 MPa to about 20 MPa, from about 0.1 MPa to about 10 MPa, from about 0.1 MPa to about 5 MPa, from about 0.2 MPa to about 4 MPa, or from about 0.3 MPa to about 2 MPa, or from about 0.5 MPa to about 1 MPa, or from about 1 MPa to about 20 MPa. In some embodiments, the ultimate tensile strength of the hollow tube, when hydrated, can be comparable to (e.g., within 15% or less) that of a soft tissue surrounding an implantation site. For instance, skin generally has an ultimate tensile strength of ˜4-˜11 MPa, muscle ˜0.1 MPa, and artery and veins ˜0.8-˜3 MPa. As used herein, the term “ultimate tensile strength,” also known as “tensile strength” or “ultimate strength,” generally refers to the maximum stress that a material can withstand while being stretched or pulled before failing or breaking. It is within one of skill in the art to determine the ultimate tensile strength of a material. In one embodiment, the ultimate tensile strength of the hollow tubes can be determined by subjecting hydrated hollow tubes to tensile tests such as uniaxial tensile tests to generate stress/strain curves, from which the ultimate tensile strength can be determined as the highest stress value attained during the tensile tests before fracture.

In some embodiments, the percent elongation of the hollow tubes, when hydrated, can be at least about 30%, at least about 40%, at least about 50%, at least about 60%, at least about 70%, at least about 80%, at least about 90% or more. In one embodiment, the hydrated hollow tubes can have a percent elongation of about 50% to about 90%. In some embodiments, the hydrated hollow tubes can have a percent elongation comparable to (e.g., within 15% or less) that of a soft tissue surrounding an implantation site. For instance, arteries and veins generally have a strain to failure of 60-100%, skin 50-90%, and muscle 60%. The percent elongation of the hollow tubes can be determined by measuring the elongation of the hollow tubes at rupture relative to the initial gauge length. Generally, a higher percent elongation value indicates a more flexible material. As used herein, the term “flexible” generally refers to a material being capable of bending or flexing such that it is pliant and yieldable in response to a change in surrounding condition (e.g., an applied force), without causing any macroscopic breaking. A flexible material can generally alter geometric shape and/or structure to accommodate a change in surrounding condition and to conform to the shape of an object brought in contact with it without losing its integrity. Thus, the term “flexible” when used in reference to a hollow tube of the biopolymer device described herein refers to a hollow tube being capable of altering its geometric shape and structure in response to a change in surrounding condition without losing its integrity and/or causing any macroscopic breaking. For example, in some embodiments, the hollow tubes described herein are flexible enough to permit bending or flexing to accommodate the complex shape of an anatomical implantation site.

In some embodiments, not only the biopolymer devices described herein can be used in a perfusion therapy for delivery or infusion of a therapeutic agent into a target tissue to be treated, but the biopolymer devices described herein can also act as cell scaffolds to facilitate augmentation, repair and/or regeneration of the target tissue. In some embodiments, the biopolymer devices and/or hollow tubes described herein can retain their original volume upon implantation at a target site for a period of time.

By “original volume” in reference to the biopolymer devices/hollow tubes described herein is generally meant the volume of the biopolymer devices as measured immediately before they are placed into a tissue to be repaired or augmented. As used herein, the term “retain” refers to maintaining the volume (e.g., size and/or shape) of at least a portion of the hollow tubes of the biopolymer devices described herein over a period of time. In some embodiments, the hollow tubes can retain over a period of time at least about 20% of their original volume, including, for example, at least about 30%, at least about 40%, at least about 50%, at least about 60%, at least about 70%, at least about 80%, at least about 90% of their original volume or higher. In some embodiments, the hollow tubes can retain 100% of their original volume, e.g., no detectable changes in the volume, within the tissue to be repaired or augmented for a period of time. In some embodiments, the volume of the biopolymer devices/hollow tubes placed into a tissue can be determined from explants, e.g., weight measurements and/or volume displacement. In one embodiment, the volume of the biopolymer devices/hollow tubes placed into a tissue can be monitored and/or measured by imaging.

The hollow tubes can retain at least a portion of their original volume for any period of time, e.g., weeks, months, or years. In some embodiments, the biopolymer devices/hollow tubes can retain, e.g., at least about 50% of their original volume (including e.g., at least about 60%, at least about 70%, at least about 80%, or higher, of their original volume) for at least about 6 weeks, at least about 7 weeks, at least about 8 weeks, at least about 3 months, at least about 4 months, at least about 5 months, at least about 6 months, at least about 7 months, at least about 8 months, at least about 9 months, at least about 10 months, at least about 11 months, at least about 1 year, at least about 2 years, at least 3 years, at least about 4 years, at least 5 years or longer. In certain embodiments, the biopolymer devices/hollow tubes can retain, e.g., at least about 70% of their original volume or higher, for at least about 3 months or longer, including, e.g., at least about 6 months, at least about 9 months, at least about 12 months or longer. In other embodiments, there can be no significant changes in the volume of the biopolymer devices/hollow tubes after placed into a tissue to be repaired or augmented for at least about 3 months or longer.

The volume retention of the biopolymer devices and/or hollow tubes can also be characterized by, e.g., degradation profile. Generally, the slower the biopolymer devices and/or hollow tubes degrade, the longer the biopolymer devices and/or hollow tubes can retain their original volume in a tissue. As used herein, the term “degrade” or “degradation” refers to a decrease in volume or size of the biopolymer device and/or hollow tube described herein. The degradation of the biopolymer device and/or hollow tube described herein can occur via cleavage of the silk fibroin matrix present in the hollow tubes into smaller fragments and/or dissolution of the silk fibroin matrix or fragments thereof. In some embodiments, the hollow tubes comprising a silk fibroin matrix can be adapted to degrade no more than 80% of their original volume, including, for example, no more than 70%, no more than 60%, no more than 50%, no more than 40%, no more than 30%, no more than 20%, no more than 10% of their original volume or lower. In some embodiments, the hollow tubes comprising a silk fibroin matrix can exhibit no significant degradation (e.g., no detectable changes in the volume) within the tissue to be repaired or augmented. In one embodiment, the hollow tubes comprising a silk fibroin matrix can be adapted to degrade no more than 50% of their original volume within the tissue to be repaired or augmented for a period of time. In one embodiment, the hollow tubes comprising a silk fibroin matrix can be adapted to degrade no more than 40% of their original volume within the tissue to be repaired or augmented for a period of time. In one embodiment, the hollow tubes comprising a silk fibroin matrix can be adapted to degrade no more than 30% of their original volume within the tissue to be repaired or augmented for a period of time. In one embodiment, the hollow tubes comprising a silk fibroin matrix can be adapted to degrade no more than 20% of their original volume within the tissue to be repaired or augmented for a period of time. In one embodiment, the hollow tubes comprising a silk fibroin matrix can be adapted to degrade no more than 10% of their original volume within the tissue to be repaired or augmented for a period of time.

The hollow tubes comprising a silk fibroin matrix can be adapted to degrade at any rate. In some embodiments, the hollow tubes comprising a silk fibroin matrix can be adapted to degrade at least a portion of their original volume over any period of time, e.g., days, weeks, months, or years. In some embodiments, the hollow tubes comprising a silk fibroin matrix can be used to deliver a fluid (optionally comprising an agent) to a target site for a desirable period of time (e.g., ranging from day(s) to week(s) to month(s)) and then degrade gradually after the application. In some embodiments, the hollow tubes comprising a silk fibroin matrix can degrade, e.g., at least about 10% of their original volume (including e.g., at least about 20%, at least about 30%, at least about 40%, at least about 50%, at least about 60%, at least about 70%, at least about 80%, at least about 90%, at least about 95% or more, of their original volume) over a period of at least about 1 day or longer, including, e.g., at least about 2 days, at least about 3 days, at least about 4 days, at least about 5 days, at least about 6 days, at least about 1 week or longer. In some embodiments, In some embodiments, the hollow tubes comprising a silk fibroin matrix can degrade, e.g., at least about 10% of their original volume (including e.g., at least about 20%, at least about 30%, at least about 40%, at least about 50%, at least about 60%, at least about 70%, at least about 80%, at least about 90%, at least about 95% or more, of their original volume) over a period of at least about 1 week or longer, including, e.g., at least about 2 weeks, at least about 3 weeks, at least about 4 weeks, at least about 5 weeks, at least about 6 weeks, at least about 7 weeks, at least about 8 weeks, at least about 3 months, at least about 4 months, at least about 5 months, at least about 6 months, at least about 7 months, at least about 8 months, at least about 9 months, at least about 10 months, at least about 11 months, at least about 1 year, at least about 2 years, at least about 3 years, at least about 4 years, at least about 5 years or longer.

In some embodiments, the hollow tubes comprising a silk fibroin matrix can be adapted to degrade, e.g., no more than 50% of their original volume (including e.g., no more than 40%, no more than 30%, no more than 20% or lower, of their original volume) in at least about 6 weeks, at least about 7 weeks, at least about 8 weeks, at least about 3 months, at least about 4 months, at least about 5 months, at least about 6 months, at least about 7 months, at least about 8 months, at least about 9 months, at least about 10 months, at least about 11 months, at least about 1 year, at least about 2 years, at least about 3 years, at least about 4 years, at least about 5 years or longer. In certain embodiments, the hollow tubes comprising a silk fibroin matrix can be adapted to degrade, e.g., no more than 30% of their original volume or lower, in at least about 3 months or longer. In other embodiments, there can be no significant degradation (i.e., no detectable changes in the volume of the hollow tubes comprising a silk fibroin matrix) after placed into a tissue to be repaired or augmented for at least about 3 months or longer. In some embodiments, the hollow tubes comprising a silk fibroin matrix can be adapted to degrade, e.g., no more than 30% of their original volume or lower, in at least about 6 months or longer (including, e.g., at least about 9 months, at least about 12 months, at least about 18 months or longer). In other embodiments, there can be no significant degradation (i.e., no detectable changes in the volume of the hollow tubes comprising a silk fibroin matrix) after placed into a tissue to be repaired or augmented for at least about 6 months or longer. In particular embodiments, the hollow tubes comprising a silk fibroin matrix can be adapted to degrade no more than 80% of their original volume or lower in at least about 1 year or longer (including, for example, at least about 2 years, at least about 3 years, at least about 4 years, at least about 5 years or longer). In some embodiments, the hollow tubes comprising a silk fibroin matrix can be adapted to degrade no more than 50% of their original volume or lower in at least about 1 year or longer.

The same or similar formulation of the hollow tubes comprising a silk fibroin matrix can manifest different responses in a subject. By way of example only, the volume retention or degradation rate of the hollow tubes comprising a silk fibroin matrix in a tissue can vary from one subject to another, e.g., because of different tissue microenvironment such as species and/or levels of various proteins or enzymes (e.g., proteolytic enzymes) present in the tissue.

In some embodiments, the hollow tubes comprising a silk fibroin matrix can be adapted to maintain a constant volume retention rate and/or degradation rate over a period of time. In some embodiments, the hollow tubes comprising a silk fibroin matrix can be adapted to have a volume retention rate or degradation rate varying with time. For example, the hollow tubes comprising a silk fibroin matrix can be coated with a polymeric material, e.g., silk fibroin of a different concentration and/or a different biodegradable and biocompatible polymer. Such coating can possess a different function and/or a different degradation rate from that of the hollow tube matrix. By way of example only, the coating of the hollow tubes comprising a silk fibroin matrix can contain at least one active agent and be adapted to degrade at a different rate (e.g., at a faster rate) from that of the hollow tubes comprising a silk fibroin matrix. Thus, upon placing the hollow tubes comprising a silk fibroin matrix in a tissue, the coating of the hollow tubes comprising a silk fibroin matrix can be adapted to degrade faster, e.g., to release the active agent for relieving the pain and/or promoting the wound healing, while the hollow tubes comprising a silk fibroin matrix can retain their volume for a longer period of time for use in perfusion therapy.

In some embodiments, the hollow tubes comprising a silk fibroin matrix can be adapted to degrade at a rate of tissue growth or regeneration.

In some embodiments, the hollow tubes comprising a silk fibroin matrix can be tuned to degrade upon completion of a perfusion therapy.

### METHODS of Making the Hollow Tubes Described Herein

In accordance with various aspects described herein, the hollow tube comprises a silk matrix. As used herein, the phrases “silk matrix” generally refers to a matrix comprising silk fibroin. In some embodiments, the silk matrix can exclude sericin. In some embodiments, the silk matrix can comprise silk fibroin, silk sericin or a combination thereof. The term “silk matrix” refer to a matrix or composition in which silk fibroin constitutes at least about 1% (w/v or w/w)(e.g., 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, or more) of the total silk matrix composition. In some embodiments, the silk matrix constitutes at least about 30%, at least about 40%, at least about 50%, at least about 60%, at least about 70%, at least about 80%, at least about 90%, at least about 95%, up to and including 100% or any percentages between about 30% and about 100%, of the total silk matrix composition.

As used herein, the term “silk fibroin” or “fibroin” refers to silk fibroin protein. The silk fibroin protein includes silk fibroin derived from silkworm silk and insect or spider silk protein. See e.g., Lucas et al., Adv. Protein Chem. 1958, 13, 107-242. In some embodiments, silk fibroin refers to a matrix (e.g., a solution, a gel, or any solid-state matrix) containing a dissolved silkworm silk fibroin or spider silk fibroin. For example, in some embodiments, silk fibroins can be obtained from cocoons of silkworms of any species, e.g., but not limited to, Bombyx mori. Accordingly, in some embodiments, silk fibroin refers to a matrix (e.g., a solution, a gel, or any solid-state matrix) obtained from degummed cocoons of silkworms. In some embodiments, silk fibroin refers to silk fibroin that is reconstituted by a process comprising dissolving degummed cocoons of silkworms of any species, e.g., but not limited to Bombyx mori. Any type of silk fibroin can be used according to aspects of the present invention. There are many different types of silk fibroin produced by or derived from a wide variety of species, including, without limitation: Antheraea mylitta; Antheraea pernyi; Antheraea yamamai; Galleria mellonella; Bombyx mori; Bombyx mandarina; Galleria mellonella; Nephila clavipes; Nephila senegalensis; Gasteracantha mammosa; Argiope aurantia; Araneus diadematus; Latrodectus geometricus; Araneus bicentenarius; Tetragnatha versicolor; Araneus ventricosus; Dolomedes tenebrosus; Euagrus chisoseus; Plectreurys tristis; Argiope trifasciata; and Nephila madagascariensis. Other silk fibroin can be derived from transgenic silk, genetically engineered silks (recombinant silk), such as silks from bacteria, yeast, mammalian cells, transgenic animals, or transgenic plants, and variants thereof. See for example, WO 97/08315 and U.S. Pat. No. 5,245,012, content of both of which is incorporated herein by reference in its entirety. In some embodiments, silk fibroin can be derived from other sources such as spiders, other silkworms, bees, synthesized silk-like peptides, and bioengineered variants thereof. In some embodiments, silk fibroin can be extracted or derived from a gland of silkworm or transgenic silkworms. See for example, WO2007/098951, content of which is incorporated herein by reference in its entirety.

In some embodiments, the silk matrix comprises low molecular weight silk fibroin fragments, i.e., the silk matrix comprises a population of silk fibroin fragments having a range of molecular weights, characterized in that: no more than 15% of total weight of the silk fibroin fragments in the population has a molecular weight exceeding 200 kDa, and at least 50% of the total weight of the silk fibroin fragments in the population has a molecular weight within a specified range, wherein the specified range is between about 3.5 kDa and about 120 kDa. Without limitations, the molecular weight can be the peak average molecular weight (Mp), the number average molecular weight (Mn), or the weight average molecular weight (Mw)

As used herein, the-phrase “silk fibroin fragments” refers to polypeptides having an amino acid sequence corresponding to fragments derived from silk fibroin protein, or variants thereof. In the context of the present disclosure, silk fibroin fragments generally refer to silk fibroin polypeptides that are smaller than the naturally occurring full length silk fibroin counterpart, such that one or more of the silk fibroin fragments within a population or composition are less than 300 kDa, less than 250 kDa, less than 200 kDa, less than 175 kDa, less than 150 kDa, less than 120 kDa, less than 100 kDa, less than 90 kDa, less than 80 kDa, less than 70 kDa, less than 60 kDa, less than 50 kDa, less than 40 kDa, less than 30 kDa, less than 25 kDa, less than 20 kDa, less than 15 kDa, less than 12 kDa, less than 10 kDa, less than 9 kDa, less than 8 kDa, less than 7 kDa, less than 6 kDa, less than 5 kDa, less than 4 kDa, less than 3.5 kDa, etc. In some embodiments, “a silk matrix comprising silk fibroin fragments” encompasses a composition comprising non-fragmented (i.e., full-length) silk fibroin polypeptide, in additional to shorter fragments of silk fibroin polypeptides. Silk fibroin fragments described herein can be produced as recombinant proteins, or derived or isolated (e.g., purified) from a native silk fibroin protein or silk cocoons. In some embodiments, the silk fibroin fragments can be derived by degumming silk cocoons under a specified condition selected to produce the silk fibroin fragments having the desired range of molecular weights. Low molecular weight silk fibroin compositions are described in U.S. Provisional Application Ser. No. 61/883,732, filed on Sep. 27, 2013, content of which is incorporated herein by reference in its entirety.

In some embodiments, the silk fibroin is substantially depleted of its native sericin content (e.g., 5% (w/w) or less residual sericin in the final extracted silk). Alternatively, higher concentrations of residual sericin can be left on the silk following extraction or the extraction step can be omitted. In some embodiments, the sericin-depleted silk fibroin has, e.g., about 1% (w/w) residual sericin, about 2% (w/w) residual sericin, about 3% (w/w) residual sericin, about 4% (w/w), or about 5% (w/w) residual sericin. In some embodiments, the sericin-depleted silk fibroin has, e.g., at most 1% (w/w) residual sericin, at most 2% (w/w) residual sericin, at most 3% (w/w) residual sericin, at most 4% (w/w), or at most 5% (w/w) residual sericin. In some other embodiments, the sericin-depleted silk fibroin has, e.g., about 1% (w/w) to about 2% (w/w) residual sericin, about 1% (w/w) to about 3% (w/w) residual sericin, about 1% (w/w) to about 4% (w/w), or about 1% (w/w) to about 5% (w/w) residual sericin. In some embodiments, the silk fibroin is entirely free of its native sericin content. As used herein, the term “entirely free” (i.e. “consisting of” terminology) means that within the detection range of the instrument or process being used, the substance cannot be detected or its presence cannot be confirmed. In some embodiments, the silk fibroin is essentially free of its native sericin content. As used herein, the term “essentially free” (or “consisting essentially of”) means that only trace amounts of the substance can be detected.

Without wishing to be bound by a theory, properties of the biopolymer devices and/or hollow tubes described herein can be modified through controlled partial removal of silk sericin or deliberate enrichment of source silk with sericin. This can be accomplished by varying the conditions, such as time, temperature, concentration, and the like for the silk degumming process.

Degummed silk can be prepared by any conventional method known to one skilled in the art. For example, B. mori cocoons are boiled for a period of time, generally about 10 to 60 minutes or longer, in an aqueous solution. In some embodiments, the cocoons can be boiled for a longer period of time, e.g., at least about 90 minutes or longer. In one embodiment, the aqueous solution is about 0.02M Na₂CO₃. The cocoons are rinsed, for example, with water to extract the sericin proteins. The degummed silk can be dried and used for preparing silk powder. Alternatively, the extracted silk can dissolved in an aqueous salt solution. Salts useful for this purpose include lithium bromide, lithium thiocyanate, calcium nitrate or other chemicals capable of solubilizing silk. In some embodiments, the extracted silk can be dissolved in about 8M-12 M LiBr solution. The salt is consequently removed using, for example, dialysis.

If necessary, the solution can then be concentrated using, for example, dialysis against a hygroscopic polymer, for example, PEG, a polyethylene oxide, amylose or sericin. In some embodiments, the PEG is of a molecular weight of 8,000-10,000 g/mol and has a concentration of about 10% to about 50% (w/v). A slide-a-lyzer dialysis cassette (Pierce, MW CO 3500) can be used. However, any dialysis system can be used. The dialysis can be performed for a time period sufficient to result in a final concentration of aqueous silk solution between about 10% to about 30%. In most cases dialysis for 2-12 hours can be sufficient. See, for example, International Patent Application Publication No. WO 2005/012606, the content of which is incorporated herein by reference in its entirety. Another method to generate a concentrated silk solution comprises drying a dilute silk solution (e.g., through evaporation or lyophilization). The dilute solution can be dried partially to reduce the volume thereby increasing the silk concentration. The dilute solution can be dried completely and then dissolving the dried silk fibroin in a smaller volume of solvent compared to that of the dilute silk solution. Alternatively or additionally, a silk solution can be concentrated by using a combination of centrifugal force, vacuum and heat to speed evaporation of the silk solution. In this embodiment, for example, the silk solution can be concentrated by using a CentriVap vacuum concentrator.

In some embodiments, the silk fibroin solution can be produced using organic solvents. Such methods have been described, for example, in Li, M., et al., J. Appl. Poly Sci. 2001, 79, 2192-2199; Min, S., et al. Sen'I Gakkaishi 1997, 54, 85-92; Nazarov, R. et al., Biomacromolecules 2004 5,718-26, content of all which is incorporated herein by reference in their entirety. An exemplary organic solvent that can be used to produce a silk solution includes, but is not limited to, hexafluoroisopropanol (HFIP). See, for example, International Application No. WO2004/000915, content of which is incorporated herein by reference in its entirety. In some embodiments, the silk solution is entirely free or essentially free of organic solvents, i.e., solvents other than water.

Generally, any amount of silk fibroin can be present in the solution used for forming the hollow tubes and/or other components of the biopolymer devices. For example, amount of silk fibroin in the solution can vary from about 0.1% (w/v) to about 90% (w/v). In some embodiments, the amount of silk fibroin in the solution can be from about 1% (w/v) to about 75% (w/v), from about 1% (w/v) to about 70% (w/v), from about 1% (w/v) to about 65% (w/v), from about 1% (w/v) to about 60% (w/v), from about 1% (w/v) to about 55% (w/v), from about 1% (w/v) to about 50% (w/v), from about 1% (w/v) to about 35% (w/v), from about 1% (w/v) to about 30% (w/v), from about 1% (w/v) to about 25% (w/v), from about 1% (w/v) to about 20% (w/v), from about 1% (w/v) to about 15% (w/v), from about 1% (w/v) to about 10% (w/v), from about 5% (w/v) to about 25% (w/v), from about 5% (w/v) to about 20% (w/v), from about 5% (w/v) to about 15% (w/v). In some embodiments, the silk fibroin solution has a silk fibroin concentration of from about 5% to about 40%, from 10% to about 40%, or from about 15% to about 40% (w/v). In some embodiments, the silk fibroin in the solution is about 25-30% (w/v). In some embodiments, the silk fibroin in the solution is about 6% (w/v) to about 10% (w/v). In some embodiments, the silk fibroin solution has a silk fibroin concentration of about 5% (w/v), about 7.5% (w/v), about 8% (w/v), about 10% (w/v), about 12.5% (w/v), about 15% (w/v), about 17.5% (w/v), about 20% (w/v), about 22.5% (w/v), about 25% (w/v), about 27.5% (w/v), about 30% (w/v), about 32.5% (w/v), about 35% (w/v), about 37.5% (w/v), about 40% (w/v), about 42.5% (w/v), about 45% (w/v), about 47.5% (w/v), or about 50% (w/v). Accordingly, the concentration of the silk solution can vary depending on the fabrication methods and/or characteristics of the hollow tubes described herein. By way of example only, in one embodiment, a hollow tube described herein can be produced by dip-coating an elongated structure (e.g., a wire) in a solution comprising silk fibroin in an amount of about 6 wt/vol % to about 10 wt/vol %. See, e.g., U.S. patent application Ser. No. 12/672,521, the content of which is incorporated herein by reference. In another embodiment, a hollow tube described herein can be produced by spinning a more concentrated silk fibroin solution (e.g., ˜25-50% (w/v)) onto a rotating and axially reciprocating elongated structure (e.g., a wire). See, e.g., U.S. patent application Ser. No. 12/934,666, the content of which is incorporated herein by reference. Exact amount of silk in the silk solution can be determined by drying a known amount of the silk solution and measuring the mass of the residue to calculate the solution concentration.

Generally, any amount of silk fibroin can be present in the hollow tubes and/or any components of the biopolymer devices disclosed herein. For example, amount of silk fibroin in the hollow tubes and/or any components of the biopolymer devices described herein (e.g., but not limited to a cap for sealing the second end of the hollow tube) can be from about 1% (w/w) to about 90% (w/w). In some embodiments, the amount of silk fibroin in the hollow tubes and/or any components of the biopolymer devices can be from about 0.1% (w/w) to about 75% (w/w), from about 1% (w/w) to about 70% (w/w), from about 1% (w/w) to about 65% (w/w), from about 1% (w/w) to about 60% (w/w), from about 1% (w/w) to about 55% (w/w), from about 1% (w/w) to about 50% (w/w), from about 1% (w/w) to about 45% (w/w), from about 1% (w/w) to about 40% (w/w), from about 1% (w/w) to about 35% (w/w), from about 1% (w/w) to about 30% (w/w), from about 1% (w/w) to about 25% (w/w), from about 1% (w/w) to about 20% (w/w), from about 1% (w/w) to about 15% (w/w), from about 1% (w/w) to about 10% (w/w), from about 5% (w/w) to about 25% (w/w), from about 5% (w/w) to about 20% (w/w), from about 5% (w/w) to about 15% (w/w). In some embodiments, the silk fibroin in the hollow tubes and/or any components of the biopolymer devices can be at least about 25% (w/w), or at least about 30% (w/w). In some embodiments, the silk fibroin in the composition can be at least about about 5 (w/w) to about 30% (w/w), about 6% (w/w) to about 20% (w/w), about 6% (w/w) to about 10% (w/w).

Without wishing to be bound by theory, molecular weight of silk or the silk fibroin concentration used for preparing the silk matrix can have an effect on properties of the silk matrix, such as swelling ratio, degradation, release kinetics, solubility, and the like

The hollow tubes comprising a silk matrix can be produced by any one skilled in the art, and thus be present in any material states or forms accordingly. For example, the hollow tubes comprising a silk matrix can be produced by a process comprising molding, dip-coating, electrospinning, gel spinning, machining, rolling a film or sheet, freeze-drying, other method known in the art, or any combinations thereof.

In some embodiments, the hollow tubes comprising a silk matrix can be made using molding, dip-coating, electrospinning, gel spinning, and the like. Gel spinning is described in Lovett et al. (Biomaterials 2008, 29(35):4650-4657) and the construction of gel-spun silk tubes is described in PCT application no. PCT/US2009/039870, filed Apr. 8, 2009, contents of both of which are incorporated herein by reference in their entireties. In one embodiment, the hollow tubes described herein can be formed by gel-spinning. For example, the hollow tubes can be formed by spinning a concentrated silk solution onto a rotating and axially reciprocating elongated structure of a desired diameter (which determines the inside diameters of the hollow tube). The silk solution can be spinned on the elongated structure, e.g., with a rotating speed of about 150-250 rpm and an axial slew rate (ASR) of about 1 mm/sec to about 3 mm/sec. The silk matrix coated on the elongated structures can then be treated to induce beta-sheet formation in silk fibroin and thus form a tubular structure, e.g., by lyophilizing the silk matrix and/or contacting the silk matrix with methanol. The silk tubes can then be removed from the elongated structures, thus forming silk fibroin-based catheters.

Construction of silk tubes using the dip-coating method is described in PCT application no. PCT/US2008/072742, filed Aug. 11, 2008, content of which is incorporated herein by reference in its entirety. In one embodiment, the hollow tubes described herein can be formed by dip-coating. By way of example only, the hollow tubes comprising silk fibroin can be generated by dip-coating elongated structures (e.g., but not limited to metal wires such as nitinol or steel wires) of a desired diameter (which determines the inside diameter of the resultant hollow tubes) into a silk fibroin solution, followed by inducing beta sheet formation in silk fibroin coated on the elongated structures, for example, in methanol (MeOH). This dipping process can be repeated to increase the wall thickness of the resultant hollow tubes. In some embodiments, the silk fibroin solution can comprise a water-soluble porogen, such as salt or PEO, to produce a porous silk tube. In these embodiments, the resultant silk tubes after beta-sheet formation can be placed into a water bath to extract the water-soluble porogen from the silk tubes and thus create pores within the silk tubes. The silk tubes were removed from the elongated structures and dried in air, forming silk fibroin-based catheters.

Construction of silk fibroin tubes using film-spinning is described in PCT application No. PCT/US2013/030206, filed Mar. 11, 2013 and U.S. Provisional application No. 61/613,185, filed Mar. 20, 2012, contents of both of which are incorporated herein by reference in their entireties.

In some embodiments of this aspect and other aspects described herein, in order to make one end of the hollow tube with a larger inside diameter or inner lumen fitting, a connector (e.g., in a form of a tubular structure) of a desired diameter, which determines the inside diameter of the hollow tube at the first end, can be assembled with or adaptably attached to the elongated structure prior to depositing a silk fibroin solution thereon. In these embodiments, the hollow tube can be formed integral to the connector, which can then be adaptably connected to a fluid delivery pump. In one embodiment, the connector can include a polypropylene tube. The polypropylene tube can have one end configured to a fluid delivery pump.

In some embodiments of this aspect and other aspects described herein where the second end of the hollow tubes is sealed, one end of the formed hollow tube can be sealed with a silk matrix by dipping one end into a concentrated silk solution and subsequently inducing beta-sheet formation in silk fibroin.

In some embodiments, the hollow tubes can be present in a form of a hydrogel comprising silk fibroin. As used herein, the term “hydrogel” refers to a swellable polymeric matrix, consisting of a three-dimensional network of macromolecules held together by covalent or non-covalent crosslinks, which can absorb a substantial amount of liquid, e.g., water, within its structure without dissolution. Exemplary methods for preparing silk fibroin gels and hydrogels include, but are not limited to, sonication, vortexing, pH titration, exposure to electric field, solvent immersion, water annealing, water vapor annealing, and the like. Exemplary methods for preparing silk fibroin gels and hydrogels are described in, for example, WO 2005/012606, WO 2008/150861, WO 2010/036992, and WO 2011/005381; and U.S. Pat. App. Pub No. U.S. 2010/0178304 and No.: US 2011/0171239, content of all of which is incorporated herein by reference in its entirety. Gels formed by exposure to electric field are also referred to as e-gels herein. Methods for forming e-gels are described in, for example, US2011/0171239, content of which is incorporated herein by reference in its entirety. Various silk fibroin gelation methods described herein can be used alone, or in combination with other methods used for forming tubular structures as noted earlier, e.g., but not limited to gel-spinning, dip-coating and/or film spinning.

In some embodiments, the hollow tube comprising silk matrix can be in the form of a sponge or foam. In some embodiments, the foam or sponge is a patterned foam or sponge, e.g., nanopatterned foam or sponge. Exemplary methods for preparing silk foams and sponges are described in, for example, WO 2004/000915, WO 2004/000255, and WO 2005/012606, content of all of which is incorporated herein by reference in its entirety. The methods for preparing silk foams and sponges can be used alone, or in combination with other methods used for forming tubular structures as noted earlier, e.g., but not limited to gel-spinning, dip-coating and/or film spinning.

In some embodiments, the hollow tube comprising silk matrix can be produced from a silk film or sheet. For example, the hollow tube comprising silk matrix can be produced by wrapping a silk film around a vertical axis to form a tubular structure. In some embodiments, the film is a patterned film, e.g., nanopatterned film. Exemplary methods for preparing silk fibroin films are described in, for example, WO 2004/000915 and WO 2005/012606, content of both of which is incorporated herein by reference in its entirety.

In some embodiments, the hollow tube can be produced by applying pressure and/or heat to short silk fibers (e.g., micron-sized silk fibers), silk particles and/or silk powder in a tubular mold to form a tubular structure. A fiber can be prepared by electrospinning a silk solution, drawing a silk solution, and the like. Electrospun silk materials, such as fibers, and methods for preparing the same are described, for example in WO2011/008842, content of which is incorporated herein by reference in its entirety. Micron-sized silk fibers (e.g., 10-600 μm in size) and methods for preparing the same are described, for example in Mandal et al., PNAS, 2012, doi: 10.1073/pnas.1119474109; U.S. Provisional Application No. 61/621,209, filed Apr. 6, 2012; and PCT application no. PCT/US13/35389, filed Apr. 5, 2013, contents of all of which are incorporated herein by reference in their entireties.

The silk particles can be of any shape or form, e.g., spherical, rod, elliptical, cylindrical, capsule, or disc. In some embodiments, the particle is a microparticle or a nanoparticle. As used herein, the term “microparticle” refers to a particle having a particle size of about 0.01 μm to about 1000 μm. In some embodiments, the microparticle as a size of about 0.05 μm to about 750 μm, about 0.1 μm to about 500 μm, about 0.25 μm to about 250 μm, or about 0.5 μm to about 100 μm. In one embodiment, the microparticle has a particle size of about 75 μm. As used herein, the term “nanoparticle” refers to particle having a particle size of about 0.1 nm to about 1000 nm. For example, a nanoparticle can have a particle size of about 0.5 nm to about 500 nm, about 1 nm to about 250 nm, about 10 nm to about 150 nm, or about 15 nm to about 100 nm.

It will be understood by one of ordinary skill in the art that microparticles or nanoparticles usually exhibit a distribution of particle sizes around the indicated “size.” Unless otherwise stated, the term “size” as used herein refers to the mode of a size distribution of microparticles or nanoparticles, i.e., the value that occurs most frequently in the size distribution. Methods for measuring the microparticle or nanoparticle size are known to a skilled artisan, e.g., by dynamic light scattering (such as photocorrelation spectroscopy, laser diffraction, low-angle laser light scattering (LALLS), and medium-angle laser light scattering (MALLS)), light obscuration methods (such as Coulter analysis method), or other techniques (such as rheology, and light or electron microscopy).

Various methods of producing silk microparticles or nanoparticles are known in the art. In some embodiments, the silk microparticles or nanoparticles can be produced by a polyvinyl alcohol (PVA) phase separation method as described in, e.g., International App. No. WO 2011/041395, the content of which is incorporated herein by reference in its entirety. Other methods for producing silk microparticles or nanoparticles are described in, for example, U.S. App. No. U.S. 2010/0028451 and International App. No.: WO 2008/118133 (using lipid as a template for making silk microspheres or nanospheres); and in Wenk et al. J Control Release 2008; 132: 26-34 (using spraying method to produce silk microspheres or nanospheres), contents of all which are incorporated herein by reference in their entireties. Certain embodiments of micro- to nano-scale silk fibroin particles and related technology are also provided in U.S. Provisional Application Ser. No. 61/883,933, filed Sep. 27, 2013, titled “SYNTHESIS OF SILK FIBROIN MICRO-AND SUBMICRON SPHERES USING A CO-FLOW METHOD,” content of which is incorporated herein by reference in its entirety.

In some embodiments, the silk particles can be reduced to smaller particles such as powder by milling, grinding, pulverizing and/or any combinations thereof.

In some embodiments, the short silk fibers, silk particles and/or silk powders can be present as reinforcing materials in the hollow tube matrix. In these embodiments, the hollow tube matrix can comprise silk-reinforcing materials in a desirable amount, e.g., ranging from about 0.1% (w/w) to about 50% (w/w), or from about 0.5% (w/w) to about 25% (w/w), or from about 1% (w/w) to about 10% (w/w), or from about 2% (w/w) to about 5% (w/w).

In some embodiments, the hollow tubes comprising a silk matrix can be a lyophilized tube or a freeze-dried tube.

Optionally, the conformation of the silk fibroin in the silk matrix can be altered after formation of the silk matrix. Without wishing to be bound by a theory, the induced conformational change can alter the crystallinity of the silk fibroin in the silk matrix, e.g., Silk II beta-sheet crystallinity. This can alter the rate of release of the therapeutic agent from the silk matrix. The conformational change can be induced by any methods known in the art, including, but not limited to, alcohol immersion (e.g., ethanol, methanol), water annealing, shear stress, ultrasound (e.g., by sonication), pH reduction (e.g., pH titration and/or exposure to an electric field) and any combinations thereof. For example, the conformational change can be induced by one or more methods, including but not limited to, controlled slow drying (Lu et al., Biomacromolecules 2009, 10, 1032); water annealing (Jin et al., 15 Adv. Funct. Mats. 2005, 15, 1241; Hu et al., Biomacromolecules 2011, 12, 1686); stretching (Demura & Asakura, Biotech & Bioengin. 1989, 33, 598); compressing; solvent immersion, including methanol (Hofmann et al., J Control Release. 2006, 111, 219), ethanol (Miyairi et al., J. Fermen. Tech. 1978, 56, 303), glutaraldehyde (Acharya et al., Biotechnol J. 2008, 3, 226), and 1-ethyl-3-(3-dimethyl aminopropyl) carbodiimide (EDC) (Bayraktar et al., Eur J Pharm Biopharm. 2005, 60, 373); pH adjustment, e.g., pH titration and/or exposure to an electric field (see, e.g., U.S. Patent App. No. US2011/0171239); heat treatment; shear stress (see, e.g., International App. No.: WO 2011/005381), ultrasound, e.g., sonication (see, e.g., U.S. Patent Application Publication No. U.S. 2010/0178304 and International App. No. WO2008/150861); and any combinations thereof. Content of all of the references listed above is incorporated herein by reference in their entirety.

In some embodiments, the conformation of the silk fibroin can be altered by water annealing. Without wishing to be bound by theory, it is believed that physical temperature-controlled water vapor annealing (TCWVA) provides a simple and effective method to obtain refined control of the molecular structure of silk biomaterials. The silk materials can be prepared with control of crystallinity, from a low content using conditions at 4° C. (a helix dominated silk I structure), to highest content of ˜60% crystallinity at 100° C. (β-sheet dominated silk II structure). This physical approach covers the range of structures previously reported to govern crystallization during the fabrication of silk materials, yet offers a simpler, green chemistry, approach with tight control of reproducibility. Temperature controlled water vapor annealing is described, for example, in Hu et al., Biomacromolecules, 2011, 12, 1686-1696, content of which is incorporated herein by reference in its entirety.

In some embodiments, alteration in the conformation of the silk fibroin can be induced by immersing in alcohol, e.g., methanol, ethanol, etc. The alcohol concentration can be at least 10%, at least 20%, at least 30%, at least 40%, at least 50%, at least 60%, at least 70%, at least 80%, at least 90% or 100%. In some embodiment, alcohol concentration is 100%. If the alteration in the conformation is by immersing in a solvent, the silk composition can be washed, e.g., with solvent/water gradient to remove any of the residual solvent that is used for the immersion. The washing can be repeated one, e.g., one, two, three, four, five, or more times.

Alternatively, the alteration in the conformation of the silk fibroin can be induced with shear stress. The shear stress can be applied, for example, by passing the silk matrix through a needle. Other methods of inducing conformational changes include applying an electric field, applying pressure, or changing the salt concentration.

The treatment time for inducing the conformational change can be any period of time to provide a desired silk II (beta-sheet crystallinity) content. In some embodiments, the treatment time can range from about 1 hour to about 12 hours, from about 1 hour to about 6 hours, from about 1 hour to about 5 hours, from about 1 hour to about 4 hours, or from about 1 hour to about 3 hours. In some embodiments, the sintering time can range from about 2 hours to about 4 hours or from 2.5 hours to about 3.5 hours.

When inducing the conformational change is by solvent immersion, treatment time can range from minutes to hours. For example, immersion in the solvent can be for a period of at least about 15 minutes, at least about 30 minutes, at least about 1 hour, at least about 2 hours, at least 3 hours, at least about 6 hours, at least about 18 hours, at least about 12 hours, at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, or at least about 14 days. In some embodiments, immersion in the solvent can be for a period of about 12 hours to about seven days, about 1 day to about 6 days, about 2 to about 5 days, or about 3 to about 4 days.

After the treatment to induce the conformational change, silk fibroin can comprise a silk II beta-sheet crystallinity content of at least about 5%, at least about 10%, at least about 20%, at least about 30%, at least about 40%, at least about 50%, at least about 60%, at least about 70%, at least about 80%, at least about 90%, or at least about 95% but not 100% (i.e., all the silk is present in a silk II beta-sheet conformation). In some embodiments, silk is present completely in a silk II beta-sheet conformation, i.e., 100% silk II beta-sheet crystallinity.

In some embodiments, the silk fibroin in the hollow tubes and/or other components of the biopolymer devices described herein can have a protein structure that substantially includes β-turn and β-strand regions. Without wishing to be bound by a theory, the silk β sheet content can impact function and in vivo longevity of the composition. It is to be understood that composition including non-β sheet content (e.g., e-gels) can also be utilized. In aspects of these embodiments, the silk fibroin in the hollow tubes and/or other components of the biopolymer devices can have a protein structure including, e.g., about 10% β-turn and β-strand regions, about 20% β-turn and β-strand regions, about 30% β-turn and β-strand regions, about 40% β-turn and β-strand regions, about 50% β-turn and β-strand regions, about 60% β-turn and β-strand regions, about 70% β-turn and β-strand regions, about 80% β-turn and β-strand regions, about 90% β-turn and β-strand regions, or about 100% β-turn and β-strand regions. In other aspects of these embodiments, the silk fibroin in the hollow tubes and/or other components of the biopolymer devices can have a protein structure including, e.g., at least 10% β-turn and β-strand regions, at least 20% β-turn and β-strand regions, at least 30% β-turn and β-strand regions, at least 40% β-turn and β-strand regions, at least 50% β-turn and β-strand regions, at least 60% (3-turn and β-strand regions, at least 70% (3-turn and β-strand regions, at least 80% β-turn and β-strand regions, at least 90% β-turn and β-strand regions, or at least 95% β-turn and β-strand regions. In yet other aspects of these embodiments, the silk fibroin in the hollow tubes and/or other components of the biopolymer devices has a protein structure including, e.g., about 10% to about 30% β-turn and β-strand regions, about 20% to about 40% β-turn and β-strand regions, about 30% to about 50% β-turn and β-strand regions, about 40% to about 60% β-turn and β-strand regions, about 50% to about 70% β-turn and β-strand regions, about 60% to about 80% β-turn and β-strand regions, about 70% to about 90% β-turn and β-strand regions, about 80% to about 100% β-turn and β-strand regions, about 10% to about 40% β-turn and β-strand regions, about 30% to about 60% β-turn and β-strand regions, about 50% to about 80% β-turn and β-strand regions, about 70% to about 100% β-turn and β-strand regions, about 40% to about 80% β-turn and β-strand regions, about 50% to about 90% β-turn and β-strand regions, about 60% to about 100% β-turn and β-strand regions, or about 50% to about 100% β-turn and β-strand regions. In some embodiments, silk β sheet content, from less than 10% to ˜55% can be used in the hollow tubes and/or other components of the biopolymer devices.

In some embodiments, the silk fibroin in the hollow tubes and/or other components of the biopolymer devices described herein can have a protein structure that is substantially-free of α-helix and random coil regions. In aspects of these embodiments, the silk fibroin in the hollow tubes and/or other components of the biopolymer devices has a protein structure including, e.g., about 5% α-helix and random coil regions, about 10% α-helix and random coil regions, about 15% α-helix and random coil regions, about 20% α-helix and random coil regions, about 25% α-helix and random coil regions, about 30% α-helix and random coil regions, about 35% α-helix and random coil regions, about 40% α-helix and random coil regions, about 45% α-helix and random coil regions, or about 50% α-helix and random coil regions. In other aspects of these embodiments, the silk fibroin in the hollow tubes and/or other components of the biopolymer devices has a protein structure including, e.g., at most 5% α-helix and random coil regions, at most 10% α-helix and random coil regions, at most 15% α-helix and random coil regions, at most 20% α-helix and random coil regions, at most 25% α-helix and random coil regions, at most 30% α-helix and random coil regions, at most 35% α-helix and random coil regions, at most 40% α-helix and random coil regions, at most 45% α-helix and random coil regions, or at most 50% α-helix and random coil regions. In yet other aspects of these embodiments, the silk fibroin in the hollow tubes and/or other components of the biopolymer devices has a protein structure including, e.g., about 5% to about 10% α-helix and random coil regions, about 5% to about 15% α-helix and random coil regions, about 5% to about 20% α-helix and random coil regions, about 5% to about 25% α-helix and random coil regions, about 5% to about 30% α-helix and random coil regions, about 5% to about 40% α-helix and random coil regions, about 5% to about 50% α-helix and random coil regions, about 10% to about 20% α-helix and random coil regions, about 10% to about 30% α-helix and random coil regions, about 15% to about 25% α-helix and random coil regions, about 15% to about 30% α-helix and random coil regions, or about 15% to about 35% α-helix and random coil regions.

In some embodiments, the silk fibroin can be modified for different applications and/or desired mechanical or chemical properties (e.g., to facilitate formation of a gradient of a therapeutic agent in silk fibroin matrices). One of skill in the art can select appropriate methods to modify silk fibroins, e.g., depending on the side groups of the silk fibroins, desired reactivity of the silk fibroin and/or desired charge density on the silk fibroin. In one embodiment, modification of silk fibroin can use the amino acid side chain chemistry, such as chemical modifications through covalent bonding, or modifications through charge-charge interaction. Exemplary chemical modification methods include, but are not limited to, carbodiimide coupling reaction (see, e.g., U.S. Patent Application. No. US 2007/0212730), diazonium coupling reaction (see, e.g., U.S. Patent Application No. US 2009/0232963), avidin-biotin interaction (see, e.g., International Application No.: WO 2011/011347) and pegylation with a chemically active or activated derivatives of the PEG polymer (see, e.g., International Application No. WO 2010/057142).

Silk fibroin can also be modified through gene modification to alter functionalities of the silk protein (see, e.g., International Application No. WO 2011/006133). For instance, the silk fibroin can be genetically modified, which can provide for further modification of the silk such as the inclusion of a fusion polypeptide comprising a fibrous protein domain and a mineralization domain, which can be used to form an organic-inorganic composite. See WO 2006/076711. In some embodiments, the silk fibroin can be genetically modified to be fused with a protein, e.g., a therapeutic protein. Additionally, the silk fibroin matrix can be combined with a chemical, such as glycerol, that, e.g., affects flexibility and/or solubility of the matrix. See, e.g., WO 2010/042798, Modified Silk films Containing Glycerol.

In some embodiments, the silk fibroin can be modified with positively/negatively charged peptides or polypeptides, such poly-lysine and poly-glutamic acid. While possible, it is not required that every single silk fibroin molecule in the composition be modified with a positively/negatively charged molecule. Methods of derivatizing or modifying silk fibroin with charged molecules are described in, for example, PCT application no. PCT/US2011/027153, filed Mar. 4, 2011, content of which is incorporated herein by reference in its entirety.

Ratio of modified silk fibroin to unmodified silk fibroin can be adjusted to optimize one or more desired properties of the composition, such as release rate or release kinetics of an agent, degradation rate of the silk fibroin matrix, and the like. Accordingly, in some embodiments, ratio of modified to unmodified silk fibroin in the composition can range from about 1000:1 (w/w) to about 1:1000 (w/w), from about 500:1 (w/w) to about 1:500 (w/w), from about 250:1 (w/w) to about 1:250 (w/w), from about 200:1 (w/w) to about 1:200 (w/w), from about 25:1 (w/w) to about 1:25 (w/w), from about 20:1 (w/w) to about 1:20 (w/w), from about 10:1 (w/w) to about 1:10 (w/w), or from about 5:1 (w/w) to about 1:5 (w/w).

In some embodiments, the silk matrix in the hollow tubes can be porous, wherein the silk matrix can have a porosity of at least about 30%, at least about 40%, at least about 50%, at least about 60%, at least about 70%, at least about 80%, at least about 90%, or higher. Too high porosity can yield a silk matrix with lower mechanical properties, but with faster release or diffusion of an agent therethrough. However, too low porosity can decrease the release or diffusion of an agent therethrough. One of skill in the art can adjust the porosity accordingly, based on a number of factors such as, but not limited to, desired release rates, molecular size and/or diffusion coefficient of the therapeutic agent, and/or concentrations and/or amounts of silk fibroin in a silk matrix.

The porous silk matrix can have any pore size. In some embodiments, the pores of a silk matrix can have a size distribution ranging from about 50 nm to about 100 μm, from about 100 nm to about 75 μm, from about 250 nm to about 50 μm, from about 0.5 μm to about 25 μm, from 1 μm to about 10 μm. As used herein, the term “pore size” refers to a diameter or an effective diameter of the cross-sections of the pores. The term “pore size” can also refer to an average diameter or an average effective diameter of the cross-sections of the pores, based on the measurements of a plurality of pores. The effective diameter of a cross-section that is not circular equals the diameter of a circular cross-section that has the same cross-sectional area as that of the non-circular cross-section. In some embodiments, the silk fibroin can be swollen when the silk fibroin scaffold is hydrated. The sizes of the pores or the mesh size can then change depending on the water content in the silk fibroin. The pores can be filled with a fluid such as water or air.

Methods for forming pores in a silk matrix are known in the art, e.g., porogen-leaching method, freeze-drying method, and/or gas-forming method. Such methods are described, e.g., in U.S. Pat. App. Nos.: US 2010/0279112, US 2010/0279112, and U.S. Pat. No. 7,842,780, the contents of which are incorporated herein by reference in their entirety.

Methods for forming pores in silk fibroin-based matrix are known in the art and include, but are not limited, porogen-leaching methods, freeze-drying methods, and/or gas-forming method. Exemplary methods for forming pores in a silk fibroin-based material are described, for example, in U.S. Pat. App. Pub. Nos.: US 2010/0279112 and US 2010/0279112; U.S. Pat. No. 7,842,780; and PCT application publication no. WO2004062697, contents of all of which are incorporated herein by reference in their entireties.

Accordingly, any desirable release rates or profiles of an agent across the wall of a hollow tube comprising a silk matrix can be, at least partly, adjusted by varying silk processing methods, e.g., concentration of silk in a silk matrix, amount of silk fibroin and/or beta-sheet conformation structures in a silk matrix, porosity and/or pore sizes of the silk matrix, and any combinations thereof.

In some embodiments, the hollow tube matrix and/or silk matrix therein can comprise a therapeutic agent. The therapeutic agent can be selected for various purposes, e.g., treatment of a damaged tissue, infection prevention, diagnostic or monitoring purposes and/or other purposes that facilitate implantation of the biopolymer device described herein. As used herein, the term “therapeutic agent” means a molecule, group of molecules, complex or substance administered to an organism for diagnostic, therapeutic, preventative medical, or veterinary purposes. As used herein, the term “therapeutic agent” includes a “drug.” This term include externally and internally administered topical, localized and systemic human and animal pharmaceuticals, treatments, remedies, anesthetics, antimicrobial agents, nutraceuticals, cosmeceuticals, biologicals, devices, diagnostics and contraceptives, including preparations useful in clinical and veterinary screening, prevention, prophylaxis, healing, wellness, detection, imaging, diagnosis, therapy, surgery, monitoring, cosmetics, prosthetics, forensics and the like. This term can also specifically include nucleic acids and compounds comprising nucleic acids that produce a therapeutic effect, for example deoxyribonucleic acid (DNA), ribonucleic acid (RNA), or mixtures or combinations thereof, including, for example, DNA nanoplexes, siRNA, shRNA, aptamers, ribozymes, decoy nucleic acids, antisense nucleic acids, RNA activators, and the like.

The term “therapeutic agent” also includes an agent that is capable of providing a local or systemic biological, physiological, or therapeutic effect in the biological system to which it is applied. For example, the therapeutic agent can act to control infection or inflammation, enhance cell growth and tissue regeneration, control tumor growth, act as an analgesic, promote anti-cell attachment, and enhance tissue and/or bone growth, among other functions. Other suitable therapeutic agents can include anti-viral agents, hormones, antibodies, or therapeutic proteins. Other therapeutic agents include prodrugs, which are agents that are not biologically active when administered but, upon administration to a subject are converted to biologically active agents through metabolism or some other mechanism. Additionally, the hollow tube matrix and/or silk matrix can contain combinations of two or more therapeutic agents.

A therapeutic agent can include a wide variety of different compounds, including chemical compounds and mixtures of chemical compounds, e.g., small organic or inorganic molecules; saccharides; oligosaccharides; polysaccharides; biological macromolecules, e.g., peptides, proteins, and peptide analogs and derivatives; peptidomimetics; antibodies and antigen binding fragments thereof; nucleic acids; nucleic acid analogs and derivatives; an extract made from biological materials such as bacteria, plants, fungi, or animal cells; animal tissues; naturally occurring or synthetic compositions; and any combinations thereof. In some embodiments, the therapeutic agent is a small molecule.

As used herein, the term “small molecule” can refer to compounds that are “natural product-like,” however, the term “small molecule” is not limited to “natural product-like” compounds. Rather, a small molecule is typically characterized in that it contains several carbon—carbon bonds, and has a molecular weight of less than 5000 Daltons (5 kDa), preferably less than 3 kDa, still more preferably less than 2 kDa, and most preferably less than 1 kDa. In some cases it is preferred that a small molecule have a molecular weight equal to or less than 700 Daltons.

Exemplary therapeutic agents include, but are not limited to, those found in Harrison's Principles of Internal Medicine, 13^{th} Edition, Eds. T. R. Harrison et al. McGraw-Hill N.Y., NY; Physicians' Desk Reference, 50^{th} Edition, 1997, Oradell New Jersey, Medical Economics Co.; Pharmacological Basis of Therapeutics, 8^{th} Edition, Goodman and Gilman, 1990; United States Pharmacopeia, The National Formulary, USP XII NF XVII, 1990, the complete contents of all of which are incorporated herein by reference.

Therapeutic agents include the herein disclosed categories and specific examples. It is not intended that the category be limited by the specific examples. Those of ordinary skill in the art will recognize also numerous other compounds that fall within the categories and that are useful according to the present disclosure. Examples include a radiosensitizer, a steroid, a xanthine, a beta-2-agonist bronchodilator, an anti-inflammatory agent, an analgesic agent, a calcium antagonist, an angiotensin-converting enzyme inhibitors, a beta-blocker, a centrally active alpha-agonist, an alpha-1-antagonist, an anticholinergic/antispasmodic agent, a vasopressin analogue, an antiarrhythmic agent, an antiparkinsonian agent, an antiangina/antihypertensive agent, an anticoagulant agent, an antiplatelet agent, a sedative, an ansiolytic agent, a peptidic agent, a biopolymeric agent, an antineoplastic agent, a laxative, an antidiarrheal agent, an antimicrobial agent, an antifungal agent, a vaccine, a protein, or a nucleic acid. In a further aspect, the pharmaceutically active agent can be coumarin, albumin, steroids such as betamethasone, dexamethasone, methylprednisolone, prednisolone, prednisone, triamcinolone, budesonide, hydrocortisone, and pharmaceutically acceptable hydrocortisone derivatives; xanthines such as theophylline and doxophylline; beta-2-agonist bronchodilators such as salbutamol, fenterol, clenbuterol, bambuterol, salmeterol, fenoterol; antiinflammatory agents, including antiasthmatic anti-inflammatory agents, antiarthritic antiinflammatory agents, and non-steroidal antiinflammatory agents, examples of which include but are not limited to sulfides, mesalamine, budesonide, salazopyrin, diclofenac, pharmaceutically acceptable diclofenac salts, nimesulide, naproxene, acetaminophen, ibuprofen, ketoprofen and piroxicam; analgesic agents such as salicylates; calcium channel blockers such as nifedipine, amlodipine, and nicardipine; angiotensin-converting enzyme inhibitors such as captopril, benazepril hydrochloride, fosinopril sodium, trandolapril, ramipril, lisinopril, enalapril, quinapril hydrochloride, and moexipril hydrochloride; beta-blockers (i.e., beta adrenergic blocking agents) such as sotalol hydrochloride, timolol maleate, esmolol hydrochloride, carteolol, propanolol hydrochloride, betaxolol hydrochloride, penbutolol sulfate, metoprolol tartrate, metoprolol succinate, acebutolol hydrochloride, atenolol, pindolol, and bisoprolol fumarate; centrally active alpha-2-agonists such as clonidine; alpha-1-antagonists such as doxazosin and prazosin; anticholinergic/antispasmodic agents such as dicyclomine hydrochloride, scopolamine hydrobromide, glycopyrrolate, clidinium bromide, flavoxate, and oxybutynin; vasopressin analogues such as vasopressin and desmopressin; antiarrhythmic agents such as quinidine, lidocaine, tocainide hydrochloride, mexiletine hydrochloride, digoxin, verapamil hydrochloride, propafenone hydrochloride, flecainide acetate, procainamide hydrochloride, moricizine hydrochloride, and disopyramide phosphate; antiparkinsonian agents, such as dopamine, L-Dopa/Carbidopa, selegiline, dihydroergocryptine, pergolide, lisuride, apomorphine, and bromocryptine; antiangina agents and antihypertensive agents such as isosorbide mononitrate, isosorbide dinitrate, propranolol, atenolol and verapamil; anticoagulant and antiplatelet agents such as Coumadin, warfarin, acetylsalicylic acid, and ticlopidine; sedatives such as benzodiazapines and barbiturates; ansiolytic agents such as lorazepam, bromazepam, and diazepam; peptidic and biopolymeric agents such as calcitonin, leuprolide and other LHRH agonists, hirudin, cyclosporin, insulin, somatostatin, protirelin, interferon, desmopressin, somatotropin, thymopentin, pidotimod, erythropoietin, interleukins, melatonin, granulocyte/macrophage-CSF, and heparin; antineoplastic agents such as etoposide, etoposide phosphate, cyclophosphamide, methotrexate, 5-fluorouracil, vincristine, doxorubicin, cisplatin, hydroxyurea, leucovorin calcium, tamoxifen, flutamide, asparaginase, altretamine, mitotane, and procarbazine hydrochloride; laxatives such as senna concentrate, casanthranol, bisacodyl, and sodium picosulphate; antidiarrheal agents such as difenoxine hydrochloride, loperamide hydrochloride, furazolidone, diphenoxylate hdyrochloride, and microorganisms; vaccines such as bacterial and viral vaccines; antimicrobial agents such as penicillins, cephalosporins, and macrolides, antifungal agents such as imidazolic and triazolic derivatives; and nucleic acids such as DNA sequences encoding for biological proteins, and antisense oligonucleotides.

Generally, any amount of the therapeutic agent can be loaded into the hollow tube matrix and/or silk matrix to provide a desired amount of release over a period of time. For example, from about 0.1 ng to about 1000 mg of the therapeutic agent can be loaded in the hollow tube matrix and/or silk matrix. In some embodiment, amount of therapeutic agent loaded into the hollow tube matrix and/or silk matrix is selected from the range about from 0.001% (w/w) up to 95% (w/w), preferably, from about 5% (w/w) to about 75% (w/w), and most preferably from about 10% (w/w) to about 60% (w/w) of the total composition. In some embodiments, amount of amount of the therapeutic agent loaded into the hollow tube matrix and/or silk matrix is from about 0.01% to about 95% (w/v), from about 0.1% to about 90% (w/v), from about 1% to about 85% (w/v), from about 5% to about 75% (w/v), from about 10% to about 65% (w/v), or from about 10% to about 50% (w/v), of the total composition.

In some embodiments, amount of the therapeutic agent loaded into the hollow tube matrix and/or silk matrix is from about 0.01% to about 5% (w/v), from about 0.05% to about 4% (w/v), from about 0.1% to about 2.5% (w/v), from about 0.25% to about 2% (w/v), from about 0.3% to about 1.5% (w/v), from about 0.4% to about 1% (w/v) of the total composition.

In some embodiments, the hollow tube matrix and/or silk matrix described herein can further comprise at least one biocompatible polymer, including at least two biocompatible polymers, at least three biocompatible polymers or more. Exemplary biocompatible polymers include, but are not limited to, a poly-lactic acid (PLA), poly-glycolic acid (PGA), poly-lactide-co-glycolide (PLGA), polyesters, poly(ortho ester), poly(phosphazine), poly(phosphate ester), polycaprolactone, gelatin, collagen, fibronectin, keratin, polyaspartic acid, alginate, chitosan, chitin, hyaluronic acid, pectin, polyhydroxyalkanoates, dextrans, and polyanhydrides, polyethylene oxide (PEO), poly(ethylene glycol) (PEG), triblock copolymers, polylysine, any derivatives thereof and any combinations thereof.

In some embodiments, the biocompatible polymer(s) can be integrated homogenously or heterogeneously within the bulk of the silk matrix. In other embodiments, the biocompatible polymer(s) can be coated on a surface of the silk matrix. In some embodiments, the biocompatible polymer(s) can be covalently or non-covalently linked to silk in the silk matrix. In some embodiments, the biocompatible polymer(s) can be blended with silk within the silk matrix.

The hollow tube matrix and/or silk matrix can comprise any desired amount of biocompatible polymer. For example, the hollow tube matrix and/or silk matrix can comprise from about 0.01% to about 50% of the biocompatible polymer. Amount of the biocompatible polymer can be based on weight, volume or moles of the total composition. Thus, amount of the biocompatible polymer present in the hollow tube and/or silk matrix can be weight/weight, weight/volume, volume/weight, or mole/mole. In some embodiments, amount of the biocompatible polymer(s) present in the hollow tube and/or silk matrix can range from about 0.1% to about 25% (w/v), from about 0.25% to about 20% (w/v), from about 0 0.5% to about 15% (w/v), from about 0.75% to about 10% (w/v), from about 1% to about 9% (w/v), from about 2% to about 7% (w/v), or from about 3% to about 6% (w/v).

In some embodiments, the biocompatible polymer is PEG or PEO. As used herein, the term “polyethylene glycol” or “PEG” means an ethylene glycol polymer that contains about 20 to about 2000000 linked monomers, typically about 50-1000 linked monomers, usually about 100-300. PEG is also known as polyethylene oxide (PEO) or polyoxyethylene (POE), depending on its molecular weight. Generally PEG, PEO, and POE are chemically synonymous, but historically PEG has tended to refer to oligomers and polymers with a molecular mass below 20,000 g/mol, PEO to polymers with a molecular mass above 20,000 g/mol, and POE to a polymer of any molecular mass. PEG and PEO are liquids or low-melting solids, depending on their molecular weights. PEGs are prepared by polymerization of ethylene oxide and are commercially available over a wide range of molecular weights from 300 g/mol to 10,000,000 g/mol. While PEG and PEO with different molecular weights find use in different applications, and have different physical properties (e.g. viscosity) due to chain length effects, their chemical properties are nearly identical. Different forms of PEG are also available, depending on the initiator used for the polymerization process—the most common initiator is a monofunctional methyl ether PEG, or methoxypoly(ethylene glycol), abbreviated mPEG. Lower-molecular-weight PEGs are also available as purer oligomers, referred to as monodisperse, uniform, or discrete PEGs are also available with different geometries.

As used herein, the term PEG is intended to be inclusive and not exclusive. The term PEG includes poly(ethylene glycol) in any of its forms, including alkoxy PEG, difunctional PEG, multiarmed PEG, forked PEG, branched PEG, pendent PEG (i.e., PEG or related polymers having one or more functional groups pendent to the polymer backbone), or PEG with degradable linkages therein. Further, the PEG backbone can be linear or branched. Branched polymer backbones are generally known in the art. Typically, a branched polymer has a central branch core moiety and a plurality of linear polymer chains linked to the central branch core. PEG is commonly used in branched forms that can be prepared by addition of ethylene oxide to various polyols, such as glycerol, pentaerythritol and sorbitol. The central branch moiety can also be derived from several amino acids, such as lysine. The branched poly(ethylene glycol) can be represented in general form as R(-PEG-OH)m in which R represents the core moiety, such as glycerol or pentaerythritol, and m represents the number of arms. Multiarmed PEG molecules, such as those described in U.S. Pat. No. 5,932,462, which is incorporated by reference herein in its entirety, can also be used as biocompatible polymers.

Some exemplary PEGs include, but are not limited to, PEG20, PEG30, PEG40, PEG60, PEG80, PEG100, PEG115, PEG200, PEG 300, PEG400, PEG500, PEG600, PEG1000, PEG1500, PEG2000, PEG3350, PEG4000, PEG4600, PEG5000, PEG6000, PEG8000, PEG11000, PEG12000, PEG15000, PEG 20000, PEG250000, PEG500000, PEG100000, PEG2000000 and the like. In some embodiments, PEG is of MW 10,000 Dalton. In some embodiments, PEG is of MW 100,000, i.e. PEO of MW 100,000.

In some embodiments, the hollow tube matrix and/or silk matrix can further comprise additives. Some exemplary additives include biologically or pharmaceutically active compounds. Examples of biologically active compounds include, but are not limited to: cell attachment mediators, such as collagen, elastin, fibronectin, vitronectin, laminin, proteoglycans, or peptides containing known integrin binding domains e.g. “RGD” integrin binding sequence, or variations thereof, that are known to affect cellular attachment (Schaffner P & Dard 2003 Cell Mol Life Sci. January; 60(1):119-32; Hersel U. et al. 2003 Biomaterials. November; 24(24):4385-415); biologically active ligands; and substances that enhance or exclude particular varieties of cellular or tissue ingrowth. Other examples of additive agents that enhance proliferation or differentiation include, but are not limited to, osteoinductive substances, such as bone morphogenic proteins (BMP); cytokines, growth factors such as epidermal growth factor (EGF), platelet-derived growth factor (PDGF), insulin-like growth factor (IGF-I and II) TGF-β1. As used herein, the term additive also encompasses antibodies, DNA, RNA, modified RNA/protein composites, glycogens or other sugars, and alcohols.

### Methods for Use

The systems and/or biopolymer devices described herein can be used for various applications, e.g., but not limited to, collection or transfer of a fluid for diagnostic analyses and/or delivery or infusion of an agent to a target site. In some embodiments, the biodegradable biopolymer device described herein can be implanted at a target tissue site to be treated and allow for continuous and/or variable dosing regimens of a therapeutic agent for treatment of the target tissue, without the need to remove the implanted biopolymer device after perfusion therapy is complete.

In one aspect, methods for using various systems and biopolymer devices are described herein. The method comprises implanting at least a portion of one or more systems and/or biopolymer devices described herein at a target site. For example, in some embodiments where one end is adapted for connection with external tubes and pumps, a portion of the systems and/or biopolymer devices can be implanted at the target site, leaving at least the open end of the hollow tube exposed to outside of the body for connection with external tubes and pumps. In some embodiments where the system and/or biopolymer device comprises a hollow tube and an implantable perfusion pump adaptably connected to one end of the hollow tube, the entire system and/or biopolymer device can be implanted at the target site. As used herein, the term “implanted,” and grammatically related terms, refers to the positioning of at least one or more hollow tube described herein at a particular site or location, either temporarily, semi-permanently, or permanently. The term does not require a permanent fixation of at least the hollow tube in a particular position or location.

In some embodiments, a target site can be a site to be treated or infused with a therapeutic agent, which is to be delivered by or released through the systems and/or biopolymer device described herein. In some embodiments, the target site can be ex vivo. In some embodiments, the target site can be in vivo. Exemplary in vivo target sites include, but are not limited to sites of a wound, trauma, disease, or tissue to be repaired, augmented or regenerated. In some embodiments, in vivo target sites can also include tissue void or indentation that are either naturally formed (e.g., aging) or created by surgical procedure for removal of tissue (e.g., a dermal cyst or a solid tumor), corticosteroid treatment, immunologic reaction resulting in lipoatrophy, tissue damage resulting from impact injuries or therapeutic treatment (e.g., radiotherapy or chemotherapy).

In some embodiments, the methods can further comprise introducing cells and/or scaffolding material at the target site concurrently, during, or after implantation of at least the hollow tube of the systems and/or biopolymer devices described herein. In some embodiments, the cells can be introduced by implantation or injection at the target site. In some embodiments, the cells can be introduced by delivering via the hollow tubes of the systems and/or biopolymer devices described herein. Without wishing to be bound by theory, administering the cells (e.g., stem cells or lipoaspirate) to a target site can enhance or accelerate host integration and/or tissue formation over time.

In some embodiments, the methods can further comprising growing cells surrounding the hollow tube. Without wishing to be bound by theory, at least the hollow tube of the systems and/or biopolymer device described herein can degrade as a tissue surrounding the target site remodels or regenerates. In some embodiments where the entire system and/or biopolymer device is implantable, the entire system and/or biopolymer device can degrade as a tissue surrounding the target site remodels or regenerates.

Accordingly, another aspect described herein also provides methods for augmenting, repairing or regenerating a tissue in a subject. The method comprises introducing cells at a target tissue site to be augmented, repaired or regenerated; and implanting at least a portion of the hollow tubes of one or more systems and/or biopolymer devices described herein at the target site. The systems and/or biopolymer devices can direct infusion of a fluid and/or agent that is introduced into the hollow tubes, into a tissue at or surrounding the target site, thereby improving augmentation, repair or regeneration of the tissue. In some embodiments, the tissue to augmented, repaired or regenerated can be a soft tissue. Examples of a soft tissue include, but are not limited to, brain tissue, facial tissue, breast tissue, skin tissue, and any non-bone tissues.

The cells introduced into the target site can be collected from the subject to be treated or from another subject of the same species or different species. Cells can be collected from a multitude of hosts including but not limited to human autograft tissues, or transgenic mammals. More specifically, human cells used can comprise cells selected from stem cells (e.g., adipocyte-derived stem cells), osteocytes, fibroblasts, lipocytes, assorted immunocytes, cells from lipoaspirate or any combinations thereof. In one embodiment, the cells can be derived from a lipoaspirate. In some embodiments, the cells can be blended into a carrier solution, and/or scaffolding material, e.g., injectable silk fibroin particles as described in the International Patent App. No. PCT/US12/64372 filed Nov. 9, 2012, or injectable silk fibroin foams as described in the International Patent Appl. No. PCT/US12/64471 filed: Nov. 9, 2012, prior to injection. In some embodiments, the cells can be administered prior to, concurrently with, or after implantation of the hollow tube of the system and/or biopolymer device at a target site. In some embodiments, the cells can be administered by implantation or injection. In some embodiments, the cells can be administered via the system and/or biopolymer device described herein. Without wishing to be bound by theory, the cells can secrete pro-angiogenic factors and/or growth factors at the target site. As the tissue regenerates or remodels to fill up a void or repair a defect, the implanted hollow tubes can degrade accordingly. In some embodiments, the implanted hollow tube of the system and/or biopolymer devices can integrate with the regenerated host tissue. In some embodiments where the entire biopolymer device is biodegradable, the entire biopolymer device can degrade as the tissue regenerates or remodels to fill up a void or repair a defect.

As used herein, by the term “augmenting” or “augmentation” is meant increasing, restoring, enhancing or replacing a tissue. In some embodiments, the tissue can lose its elasticity, firmness, shape and/or volume. In some embodiments, the tissue can be partially or completely lost (e.g., removal of a tissue) or damaged. In those embodiments, the term “augmenting” or “augmentation” can also refer to decreasing, reducing or alleviating at least one symptom or defect in a tissue (for example, but not limited to, loss of elasticity, firmness, shape and/or volume in a tissue; presence of a void or an indentation in a tissue; loss of function in a tissue) by implanting into the tissue with at least a portion of one system and/or biopolymer device described herein. In such embodiments, at least one symptom or defect in a tissue can be decreased, reduced or alleviated by at least about 30%, at least about 40%, at least about 50%, at least about 60%, at least about 70%, at least about 80% or higher, as compared to no treatment. In some embodiments, at least one symptom or defect in a tissue can be decreased, reduced or alleviated by at least about 90%, at least about 95%, at least about 97%, or higher, as compared to no treatment. In some embodiments, at least one symptom or defect in a tissue can be decreased, reduced or alleviated by 100% (defect-free or the defect is undetectable by one of skill in the art), as compared to no treatment. In other embodiments, the tissue can be augmented to prevent or delay the onset of defect manifestation in a tissue, e.g., loss of elasticity, firmness, shape and/or volume in a tissue.

As used herein, the phrase “soft tissue augmentation” is generally used in reference to altering a soft tissue structure, including but not limited to, increasing, restoring, enhancing or replacing a tissue. In some embodiments, soft tissue augmentation is used to increases the strength of the soft tissue. In other embodiments, soft tissue augmentation is used to improve the cosmetic or aesthetic appearance of the soft tissue. For example, breast augmentation (also known as breast enlargement, mammoplasty enlargement, augmentation mammoplasty) alters the size and shape of a woman's breasts to improve the cosmetic or aesthetic appearance of the woman.

In some embodiments, at least the hollow tube of the systems and/or the biopolymer devices described herein can be implanted at a target site to facilitate soft tissue repair. In some embodiments, the biopolymer device implanted for soft tissue repair can be adapted to form a catheter. In some embodiments, at least the hollow tube of the systems and/or the biopolymer device implanted for tissue repair can be adapted to form a stent. The term “repair” or “repairing” as used herein, with respect to a tissue, refers to any correction, reinforcement, reconditioning, remedy, regenerating, filling of a tissue that restores volume, shape and/or function of the tissue. In some embodiments “repair” includes full repair and partial repair. For example, the volume, shape and/or function of a tissue to be repaired can be restored by at least about 30%, at least about 40%, at least about 50%, at least about 60%, at least about 70%, at least about 80% or higher, as compared to no treatment. In some embodiments, the volume, shape and/or function of a tissue to be repaired can be restored by at least about 90%, at least about 95%, at least about 97%, or higher, as compared to no treatment. In some embodiments, the volume, shape and/or function of a tissue to be repaired can be restored by 100% (defect-free or the defect is undetectable by one of skill in the art), as compared to no treatment. In various embodiments, at least a portion of the systems and/or biopolymer devices described herein can be implanted at a target site to repair any soft tissues discussed earlier, e.g., breast, skin, and any soft tissues amenable for soft tissue augmentation. In some embodiments, the term “repair” or “repairing” are used herein interchangeably with the term “regeneration” or “regenerate” when used in reference to tissue treatment.

In some embodiments, the systems and/or the biopolymer devices described herein can be used for soft tissue reconstruction. As used herein, the phrase “soft tissue reconstruction” refers to rebuilding a soft tissue structure that was severely damaged or lost, e.g., by a dramatic accident or surgical removal.

In some embodiments, the methods of use or treatment described herein can further comprise introducing a therapeutic agent into the hollow tube of the systems and/or biopolymer devices implanted at the target site. By way of example only, in some embodiments, the implanted hollow tube can be adaptably connected to an extracorporeal fluid delivery device, thereby flowing and directing into the hollow tube a therapeutic agent, which subsequently permeates across the hollow tube wall into the tissue surrounding the hollow tube. Alternatively, in some embodiments, the systems and/or biopolymer devices described herein to be implanted at the target site can comprise a hollow tube and an implantable agent delivery device adaptably connected to the hollow tube, whereby an agent can be introduced into the hollow tube at a pre-set flow rate upon implantation.

Any therapeutic agent that can promote augmentation, repair and/or regeneration of a tissue can be introduced into the hollow tube of the systems and/or biopolymer devices described herein, whereby the therapeutic agent directly and locally perfuses into the tissue and/or implanted cells surrounding the hollow tube. By way of example only, in some embodiments, at least one or more pro-angiogenic agents can be introduced into the hollow tube of the systems and/or biopolymer devices described herein. As used herein, the term “pro-angiogenic agent” is intended to mean an agent that directly or indirectly stimulates, enhances and/or stabilizes angiogenesis. Without wishing to be bound by theory, angiogenesis can facilitate transport of nutrients to and/or waste from the tissue, thereby promoting tissue growth and/or regeneration. Exemplary pro-angiogenic agents include, but are not limited to, VEGF, FGF, Ang1, Ang2, PDGF-BB, and any combinations thereof.

In some embodiments, at least one or more anti-inflammatory agent can be introduced into the hollow tube of the systems and/or biopolymer devices described herein. The term “anti-inflammatory agent,” as used herein, refers to an agent capable of counteracting the effects of pro-inflammatory and/or inflammatory agents and other agents that mediate an inflammatory condition or reaction. Examples of an anti-inflammatory agent can include, but are not limited to, inhibitors of any pro-inflammatory agents as described above, e.g., in a form of soluble receptors, receptor antagoinsts, aptamers, antibodies, or any combinations thereof; and/or an agent that can mediate an inflammatory pathway in a cell, e.g., in a form of soluble proteins, antisense oligonucleotides, siRNA, shRNA, vectors, or any combinations thereof. For example, an anti-inflammatory agent can include an agent that can inhibit a particular protein function and/or silence a specific gene that induces inflammation; or an agent that can promote a particular protein function and/or express a specific gene that inhibits inflammation. In some embodiments, an anti-inflammatory agent can be or include a steroid, a nonsteroidal anti-inflammatory drug, an analgesic, an inhibitor of at least one or more chemokines (e.g., but not limited to, CXCL-8, CCL2, CCL3, CCL4, CCL5, CCL11, and CXCL10) and/or a COX-2 inhibitor. A variety of anti-inflammatory agents are known to those skilled in the art, e.g., as described in International Patent App. NO. WO 2004/082588, the content of which is incorporated herein by reference, and can be added to a cell culture medium and/or used to stimulate or challenge tissue-specific cells and/or immune cells within the device to provoke an anti-inflammatory response.

In some embodiments, at least one or more growth-promoting agents can be introduced into the hollow tube of the systems and/or biopolymer devices described herein. As used herein, the term “growth-promoting agent” refers to an agent that stimulates cell proliferation. Examples of a growth-promoting agent can include but are not limited to any art-recognized growth factors such as Bone morphogenetic proteins (BMPs); Brain-derived neurotrophic factor (BDNF); Epidermal growth factor (EGF); Erythropoietin (EPO); Fibroblast growth factor (FGF); Glial cell line-derived neurotrophic factor (GDNF); Granulocyte colony-stimulating factor (G-CSF); Granulocyte macrophage colony-stimulating factor (GM-CSF); Hepatocyte growth factor (HGF); Hepatoma-derived growth factor (HDGF); Insulin-like growth factor (IGF); Myostatin (GDF-8); Nerve growth factor (NGF) and other neurotrophins; Platelet-derived growth factor (PDGF); Thrombopoietin (TPO); Transforming growth factor alpha(TGF-α); Transforming growth factor beta(TGF-β); Vascular endothelial growth factor (VEGF); Placental growth factor (P1GF); hormones, steroid hormones, and any combinations thereof.

By way of example only, in some embodiments where an adipose tissue is to be augmented or regenerated, at least one or more adipogenic agents can be introduced into the hollow tube of the systems and/or biopolymer devices described herein. As used herein, the term “adipogenic agents” refers to agents that can induce or enhance adipogenesis or cell differentiation to adipocytes; and/or can improve survival and/or proliferation of adipocytes. Examples of adipogenic agents include, but are not limited to, insulin, dexamethasone, IGF-1, cAMP, glucocorticoid, triiodothyronine, indomethacin, IBMX, thiazolidinediones, glitazones, rosiglitazone and similar agents, mammalian adipogenic factors described in the International Patent Appl. No. WO 1991/018924, and any combinations thereof. The content of the patent application is incorporated herein by reference. Accordingly, methods for regenerating an adipose tissue in a subject are also provided herein.

### Some Selected Definitions

For convenience, certain terms employed herein, in the specification, examples and appended claims are collected herein. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

As used herein the term “comprising” or “comprises” is used in reference to compositions, methods, and respective component(s) thereof, that are useful to an embodiment, yet open to the inclusion of unspecified elements, whether useful or not.

The singular terms “a,” “an,” and “the” include plural referents unless context clearly indicates otherwise. Similarly, the word “or” is intended to include “and” unless the context clearly indicates otherwise.

Other than in the operating examples, or where otherwise indicated, all numbers expressing quantities of ingredients or reaction conditions used herein should be understood as modified in all instances by the term “about.” The term “about” when used in connection with numeric values may mean±5% of the value being referred to. For example, about 100 means from 95 to 105.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. The terms, “patient” and “subject” are used interchangeably herein.

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “increased”,“increase” or “enhance” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means at least two standard deviation (2SD) away from a reference level. The term refers to statistical evidence that there is a difference. It is defined as the probability of making a decision to reject the null hypothesis when the null hypothesis is actually true.

As used interchangeably herein, the terms “essentially” and “substantially” mean a proportion of at least about 60%, or preferably at least about 70% or at least about 80%, or at least about 90%, at least about 95%, at least about 97% or at least about 99% or more, or any integer between 70% and 100%. In some embodiments, the terms “essentially” and “substantially” mean a proportion of at least about 90%, at least about 95%, at least about 98%, at least about 99% or more, or any integer between 90% and 100%. In some embodiments, the terms “essentially” and “substantially” do not include 100%. In some embodiments, the terms “essentially” and “substantially” can include 100%.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

The disclosure is further illustrated by the following examples which should not be construed as limiting. The examples are illustrative only, and are not intended to limit, in any manner, any of the aspects described herein. The following examples do not in any way limit the invention.

### EXAMPLES

The present Examples illustrate design, fabrication and characterization of one or more embodiments of the biodegradable and microporous silk fibroin-based catheters described herein that can circumvent both the indwelling problem such as catheter-associated infections, encrustation and/or blockage and eliminate the need to subsequently remove the implanted catheters. The silk fibroin-based catheters described herein can be inserted and maintained in vivo, e.g., for continuous drug delivery directly to target sites such as disease or repair sites, and degrade gradually over a period of time. The degradation lifetime of the silk fibroin-based catheters can be tuned to vary from days to months or years pending the mode of silk processing. See, e.g., Wang Y et al. Biomaterials 2008; 29(24-25):3415-3428. In the present Examples below, the silk fibroin-based catheters were prepared, e.g., by dip coating silk and polyethylene oxide (PEO) mixtures or gel spinning highly concentrated silk solution onto micron-sized wires. The fabricated silk tubes were morphologically analyzed using SEM and their tensile properties were assessed. In some embodiments, an implantable osmotic pump filled with a therapeutic agent (e.g., insulin or dexamethasone) was connected to the silk fibroin-based catheter in order to assess the drug release potential. The catheters were also studied in an in-vivo murine model of adipose tissue reconstruction using implantable slow-release pumps to infuse dexamethasone and/or insulin into a graft comprising lipoaspirate and an indwelling silk fibroin-based catheter.

### Example 1

### Design and Characterization of an Exemplary Porous Silk Fibroin-Based Catheter Tube Exemplary Design of a Porous Silk Fibroin-Based Catheter Tube

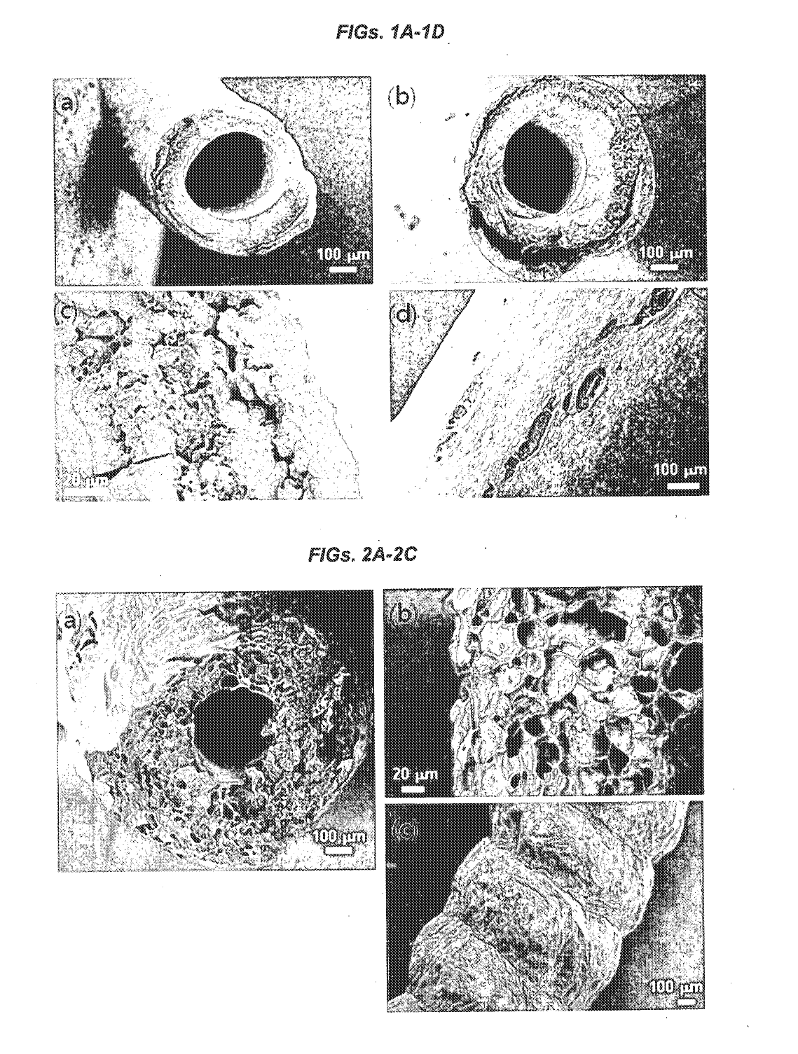

To form tailored silk fibroin-based catheters, a multiple dip-coating process was utilized, allowing deposition of silk solution onto a micron-sized metal wires. The inner diameter of silk tubes can be controlled by using different diameters of metal wires and the wall thickness of silk tube can be tuned based on altering dip-coating times and/or silk concentrations. SEM images of silk tubes generated using the multiple dip-coating method showed that each dip-coat generated a 30˜50 μm-thick wall of the silk tubes (˜380 μm inner diameter); therefore, around 100˜150 μm and 180˜250 μm thick walls were obtained depending on the numbers of dip-coatings (FIGS. 1A-1D). To generate porous structures in these tubes, in some embodiments, PEO (˜20 wt %) was mixed into the silk solution (80 wt %). After stabilizing the silk structure to form silk tubes, PEO domains within the silk tube matrix were leached out, resulting in pore formation in the silk tube matrix (FIG. 1C).

The gel spinning process was utilized for engineering silk fibroin-based catheters (FIGS. 2A-2C). A custom-designed liquid silk spinning device, as previously described in Lovett et al. 29 Biomaterials 4650 (2008) and U.S. patent application Ser. No. 12/934,666, the content of which is incorporated herein by reference, was used to deposit a highly concentrated silk solution onto a reciprocating rotating wire. The liquid silk spinning device can tailor silk tube features by controlling, e.g., winding parameters, wire size and/or post spinning processes. In the present Example, an all aqueous-based processing method was used with desired or appropriate concentration (˜25˜30 w/v %) of the aqueous silk solution. After deposition, the layers can be methanol-treated, air-dried, and/or lyophilized as part of the post-winding processing. As shown in FIGS. 2A-2C, lyophilization was used to generate porous structures within thin film layers on both inner and outer sides of the porous silk protein tubes. The silk tubes (˜380 μm in inner diameter and ˜300˜350 μm in wall thickness) were formed, e.g., by winding four layers of highly concentrated silk solution (˜25˜30 w/v %) onto a reciprocating rotating wire (˜380 μm sized metal wire), whereas the winding times and different sizes of wires can be chosen for the desired final wall thickness and inner diameter of the tubes.

In some embodiments, silk tubes can also be directly fabricated together with polypropylene tubes for connections with liquid flow (FIGS. 3A-3B). For example, the silk solution was directly gel-spun on the metal wire, of which one end was attached to a polypropylene tube, while post-processing was performed in the same way as above.

### Mechanical Properties

The tensile properties of the silk tubes in the wet state (e.g., 37° C. in PBS buffer with pH 7.4) are listed in Table 1 below.

TABLE 1
Tensile properties of silk tubes (N = 4, mean ± SD).

| | Young's modulus | Ultimate Tensile | |
|---|---|---|---|
| Silk tubes | (MPa) | strength (MPa) | Elongation (%) |
| Dip-coated tubes | 3.61 ± 0.73 | 0.64 ± 0.80 | 62.3 ± 14.7 |
| Gel spun tubes | 1.57 ± 0.17 | 0.64 ± 0.14 | 86.1 ± 22.5 |

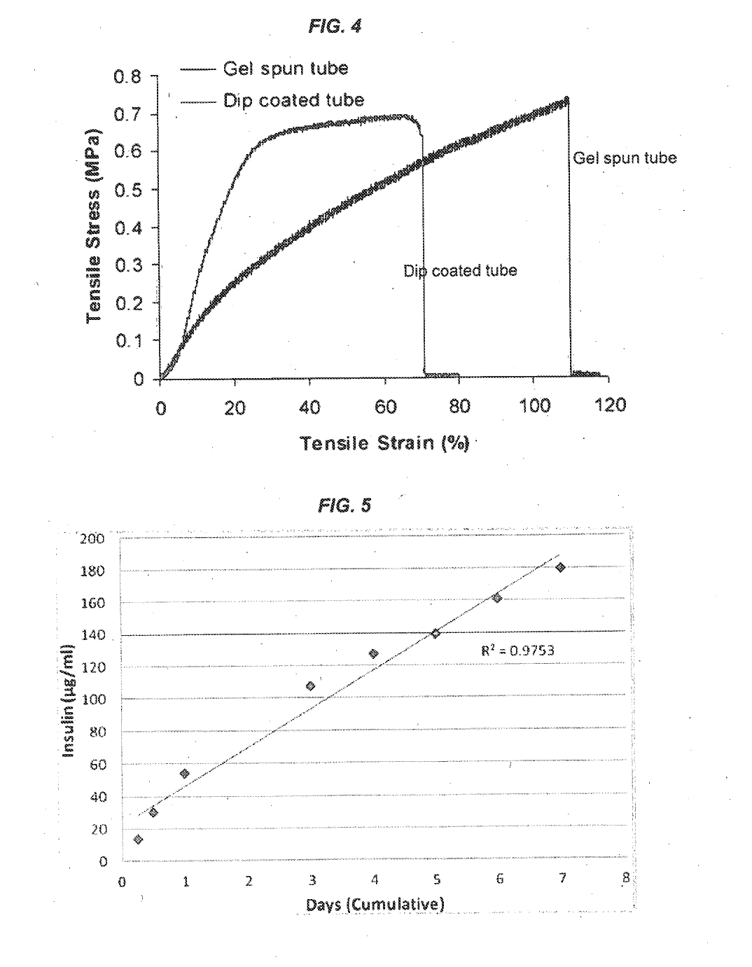

The tensile properties of the silk tubes can provide insight into their performance under physiological conditions after hypodermic insertion of the silk tubes into tissues. The strain-stress curve of the dip-coated silk tubes exhibited an elastic region at the initial stage of strain, followed by a plastic deformation region, followed by a breaking point (FIG. 4). However, the stress-strain curve of the prepared gel-spun silk tubes showed a secondary elastic region even after slightly showing a yield point around at 18% of tensile train until the breaking point (FIG. 4). Average tensile modulus, strength and elongation of the dip-coated silk tubes were 3.61±0.73 MPa, 0.64±0.80 MPa and 62.3±14.7%, respectively. Average tensile modulus, strength and elongation of the gel spun silk tubes were 1.57±0.17 MPa, 0.64±0.14 MPa and 86.1±22.5%, respectively. It is noted that the tensile modulus of the dip coated silk tube was twice that of the gel spun silk tubes, while their tensile strengths were similar. Therefore, the dip coated silk tubes were stiffer than the gel spun silk tubes, whereas they have same strength. Elongation data also showed that the gel spun silk tubes were able to be elongated to a larger extent than that of the dip coated tubes. Thus, the gel spun silk tubes in the present Example were more flexible than the dip coated silk tubes, whereas they showed same strength.

### Example 2

### In-Vitro Drug Release Analysis

The design of the silk fibroin-based catheter depicted in FIG. 3B shows the dimensions of one embodiment of the silk fibroin-based catheters used in this Example, but can be modified to suit different applications. In this embodiment, one end of the silk fibroin-based catheters was designed to connect to implantable mini-pumps, and another end of the catheter was sealed off with silk fibroin, thereby ensuring that fluid flowed through the porous wall of the silk fibroin-based catheter (FIGS. 2A-2C). In some embodiments, one end of the catheters was connected to implantable slow-release osmotic pumps (e.g., ALZET, Durect Corporation), e.g., using medical grade sealant (e.g., Dow Corning) (FIG. 8A). In order to perform the in vitro drug release analysis, the silk fibroin-based catheters, upon connection to osmotic pumps, were immersed in PBS solution at 37° C. to assess the drug release profile. An insulin solution was flown through the silk tube using the osmotic pump. FIG. 5 shows release of insulin into the PBS solution from the pores of the silk tubes over a 7-day time period. Because the silk fibroin-based catheter was sealed at the free end and at the junction of the pump/silk fibroin-based catheter, the insulin infused into the PBS solution through the pores along the length of the silk fibroin-based catheter.

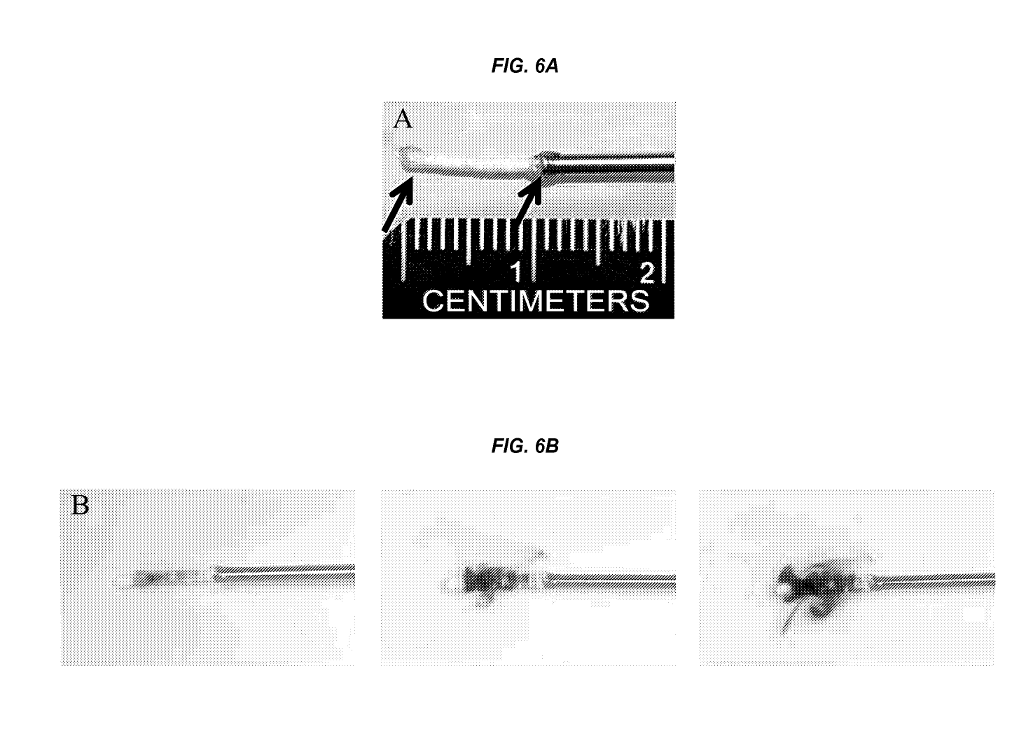

To visualize the flow of fluid through the porous length of the silk fibroin-based catheter, the silk fibroin-based catheter was connected to a blunt-tipped needle and sealed using e6000 at the distal end and at the needle/catheter junction. One drop of FD&C Blue 1 food coloring was added to 1 ml PBS and placed into a 1 ml syringe barrel. The silk fibroin-based catheter was immersed in a bath of PBS, and digital photographs were taken as the dye was slowly introduced into and then diffused out from the pores of the silk fibroin-based catheter (FIGS. 6A-6B). The dye was observed to flow from the lateral wall along the length of the silk fibroin-based catheter, and was not observed to leak from the ends of the silk fibroin-based catheter where the sealant was applied. This visualization experiment indicated that the design of the silk fibroin-based catheter allows for slow release of fluid from the pores of the catheter wall.

### Exemplary Materials and Methods Used in the Examples

Preparation of silk solution. Silk solution was generated from Bombyx mori silkworm cocoons according to the procedures previously described in Jin et al. 15 Advanced Functional Materials 1241 (2005); and Kim et al. 26 Biomaterials 2775 (2005). Cocoons of B. mori silkworm silk were obtained, for example, from Tajima Shoji Co (Yokohama, Japan). The cocoons were degummed in a boiled ˜0.02 M Na₂CO₃ (Sigma-Aldrich, St Louis, Mo.) solution for about 20 min. The fibroin extract was then rinsed three times in Milli-Q water, dissolved in a ˜9.3-M LiBr solution yielding a ˜20% (w/v) solution, and subsequently dialyzed (MWCO 3,500) against distilled water for about 2 days to obtain aqueous silk fibroin solution (ca. 8 wt/vol %).

Preparation of Silk Fibroin-Based Catheters.

Silk fibroin-based catheters were formed, for example, using multiple dip-coating (see, e.g., Lovett M et al. Biomaterials 2007; 28(35):5271-5279) or gel spinning (see, e.g., Lovett M L et al. Biomaterials 2008; 29(35):4650-4657). For multiple dip-coating, a mixture of silk fibroin (e.g., ˜10 ml of ˜8 wt/vol %) and PEO (MW=˜900,000; Sigma-Aldrich) (˜4 ml of ˜5 wt/vol %) solution (Silk:PEO ˜4:1 w/w) was prepared to form a porous silk tubular matrix. Silk tubes were generated by dip-coating nitinol wires (e.g., ˜30 μm in diameter) (McMaster, Elmhurst, Ill.) into the solution, treating the coated mixture solution on the wires, for example, in methanol (MeOH) for about 30 secs, to stabilize the silk on the wire surface, and then drying the coated layer in air for about 1 hour. This dipping process was repeated to generate around 200˜300 μm thick tubular matrices on the wires. After the dip coating process, the silk tubes were treated, e.g., in MeOH for 2 hours, and then placed into a water bath, e.g., for 2 days, to extract the PEO from the silk tubes and thus induce pore formation within the silk tubes. The silk tubes were removed from the wire and dried in air, forming silk fibroin-based catheters.

For the gel spinning process, the aqueous silk fibroin solution was further concentrated (e.g., to about 25˜30 w/v %), for example, by using a CentriVap vacuum concentrator (Labconco, Kansas City, Mo.). Tubular scaffolds were produced by spinning the concentrated silk solutions [25-30% (w/v), about 0.1 ml/˜5 cm of scaffold] onto a rotating (˜200 rpm) and axially reciprocating wire (˜380 μm (˜0.015 in) in diameter) with an axial slew rate (ASR) of about 2 mm/sec using a custom gel spinning platform and program as previously described in Lovett et al. 29 Biomaterials 4650 (2008) and U.S. patent application Ser. No. 12/934,666, the content of which is incorporated herein by reference. A polypropylene tube (e.g., ˜1090 μm outer diameter) was assembled with or adaptably attached to the wire to make one end of the resultant silk tube with a larger inner lumen fitting (FIG. 3A). The silk tubes were then lyophilized and treated, e.g., with methanol for 2 hours, and removed from the wires, thus forming silk fibroin-based catheters. One end of the silk fibroin-based catheter (e.g., ˜380 μm in diameter) was capped with a silk matrix by dipping one end into concentrated silk solution, treating, e.g., in MeOH for 2 minutes, and drying.

Scanning Electron Microscopy (SEM).

Fractured sections of the silk tubes were obtained in liquid nitrogen using a razor blade. The fracture surfaces were sputter coated with Au/Pd. The morphology was examined with a Field Emission Scanning Electron Microscope (FESEM) Zeiss MA 10 (Carl Zeiss AG, Germany) at ˜3 kV. Pore size and wall thickness of silk scaffolds were analyzed with ImagePRO Plus 6.0 (Media Cybernetics, Inc., MD).

Mechanical Analysis.

Uniaxial tensile tests were performed on an Instron 3366 testing frame equipped with a 10 N capacity load cell and Biopuls pneumatic clamps. See, e.g., Lawrence B D et al. Macromol Biosci 2010; 10(4):393-403. The silk tubes were hydrated in 0.1 M phosphate-buffered saline (PBS) for 24 h to reach swelling equilibrium prior to testing. Test samples were submerged in a temperature-controlled testing container (Biopuls) filled with PBS solution (37° C.). A displacement control mode with a crosshead displacement rate of 3 mm·min−1 was used, and the gauge length was 10 mm. The area of the gauge region were calculated by the average specimen outer diameter (measured by a Carrera Precision Digital LCD Caliper micrometer) and inner diameter (measured by SEM) to convert load data to tensile stress values. The initial elastic modulus, yield stress, and tensile strength were calculated from stress/strain plots. See, e.g., Lawrence B D et al. Macromol Biosci 2010; 10(4):393-403. The initial elastic modulus was calculated by using a least-squares (LS) fitting between 0.05N load and 5% strain past this initial load point. See, e.g., Lawrence B D et al. Macromol Biosci 2010; 10(4):393-403. Ultimate tensile strength (UTS) was determined as the highest stress value attained during the test. Results were statistically analyzed using one-way analysis of variance (ANOVA).

In-Vitro Drug Release Analysis.

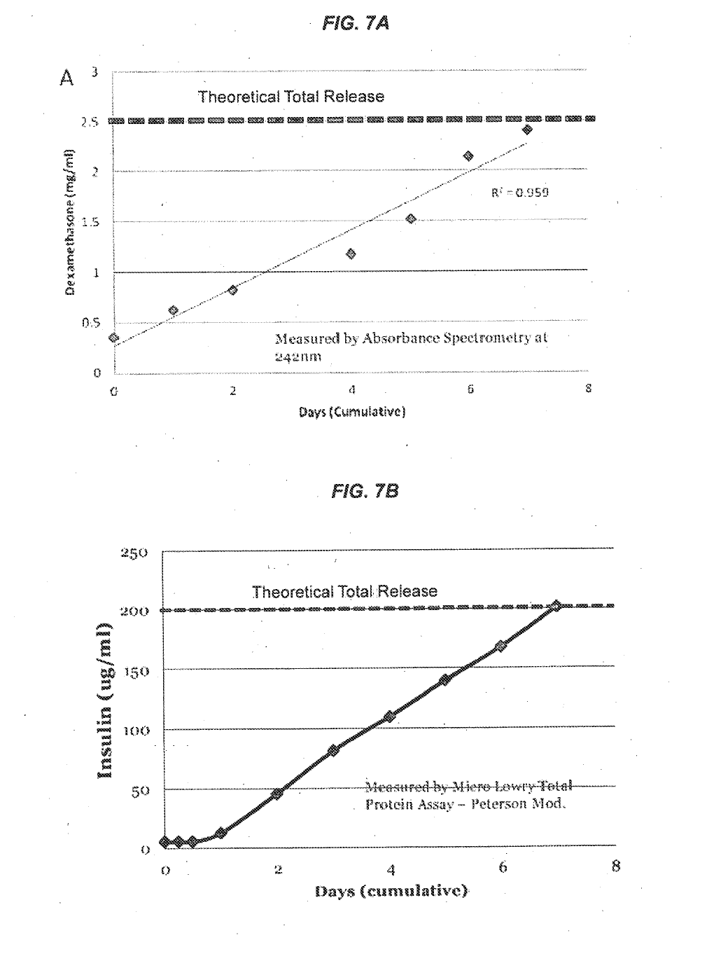

Infusion of a therapeutic agent such as insulin (in a buffered solution such as PBS) through the pores of the silk fibroin-based catheter was performed using an osmotic pump, e.g., ALZET osmotic pump (Durect Corp, Cupertino, Calif.). A ˜1-mm long silk fibroin-based catheter was connected to an osmotic pump, e.g., a 7-day release osmotic pump (with a flow rate of about 0.5 μL/hr) containing insulin (Sigma-Aldrich). The exposed end of the silk fibroin-based catheter and the junction of the catheter/pump were sealed off with a sealant to prevent leakage. The pump/catheter system was primed by immersing in ˜1 ml PBS at 37° C. for 24 hours, per manufacturer's instructions. For each timepoint (6, 12, 24 hours, and daily for 7 days), the ˜1 ml PBS/insulin sample solution was collected and stored at −80° C. for further analysis, and fresh ˜1 ml PBS was replaced. Insulin in each sample was measured using microLowry protein assay, and the data were plotted as cumulative release over time. Dexamethasone was measured using absorbance spectroscopy at ˜242 nm. Data were plotted as cumulative release over time. Both insulin and dexamethasone were continuously released from the silk fibroin-based catheter in a controlled manner (FIGS. 7A-7B).

In-vivo Animal Study.

Surgical Procedure:

Female athymic nude mice were divided into 3 groups: animals treated with insulin (n=24), dexamethasone (n=15), or PBS vehicle (n=10). The groups were further subdivided into four different doses for insulin (e.g., ˜100 μg, ˜300 μg, ˜650 μg, or ˜1000 μg) or three different doses for dexamethasone (e.g., ˜1 mg/ml, ˜2 mg/ml, ˜3 mg/ml). Osmotic pumps that can deliver a fluid with a selected flow rate for a period of time were prepared with the insulin or dexamethasone solution or PBS vehicle. In this Example, ALZET 4-week osmotic pumps with a selected flow rate of ˜0.11 μl/hr were used. One end of the silk fibroin-based catheter was connected to the pump, e.g., using medical grade silicone (e.g., Dow Corning) (FIG. 8A). The silk catheter/pump construct was primed for 48 hours at 37° C., per manufacturer's instructions.

Lipoaspirate grafts were obtained from human subcutaneous abdominal fat during scheduled elective surgery and processed for injection under laboratory conditions. Lipoaspirate was loaded into ˜1 ml syringes in sterile fashion. Mice were anesthetized with 2% isoflurane in oxygen at the time of the procedure. Once the anesthetic plane was obtained, the animals were prepped for sterile surgery. Each animal received two bilateral ˜500 μl subcutaneous injections of lipoaspirate on the dorsal region, for example, through a 16G Coleman cannula using the fan-injection technique, which uniformly spreads the fat graft created beneath the cutaneous layer. For each animal, one of the two fat grafts received a silk fibroin-based catheter connected to an implantable osmotic pump. The silk catheter/pump was implanted subcutaneously through a small incision between the scapulae, and the fat graft was placed surrounding the silk fibroin-based catheter. The incision for the catheter/pump implantation was closed with a standard wound clip. To prevent and/or reduce pain and discomfort, mice were given subcutaneous injections of ˜5 mg/kg ketoprofen (Fort Dodge Animal Health, Fort Dodge, Iowa) immediately after the surgical procedure and every 24 hours as needed.

Euthanasia and Specimen Harvest:

At 4 weeks post surgery, the mice were euthanized using carbon dioxide asphyxiation followed by cervical dislocation, and graft tissue was isolated from the bilateral dorsal injection sites. Explants were assessed for mass on a standard laboratory balance, and volume was measured using a gas pycnometer (Accupyc II 1340; Micrometrics, Atlanta, Ga.). Immediately after the dimensional analysis was complete, the tissue samples were fixed in 10% neutral buffered formalin and processed and embedded in paraffin.

Results:

For the dexamethasone-treated animals there were no adverse events during the therapy. For the insulin-treated animals, the fat graft appears to maintain healthy with no evidence of oil cysts or adverse biocompatibility reaction, except that the groups of animals receiving highest insulin (˜1000 μg group and ˜650 μg) generally died during the study period.

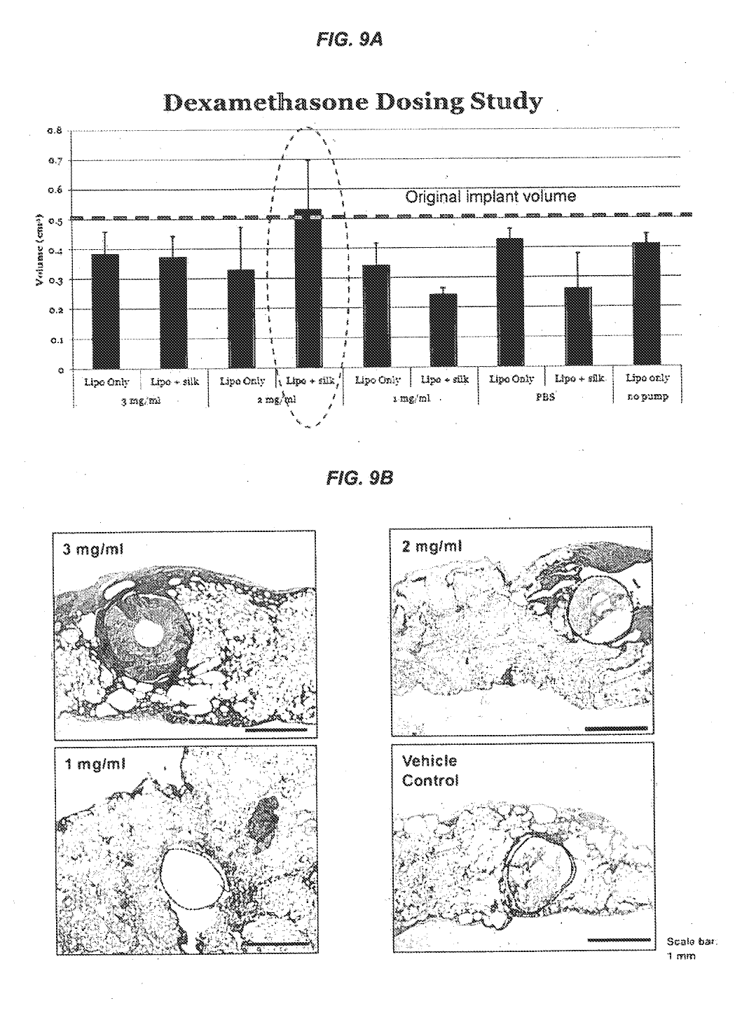

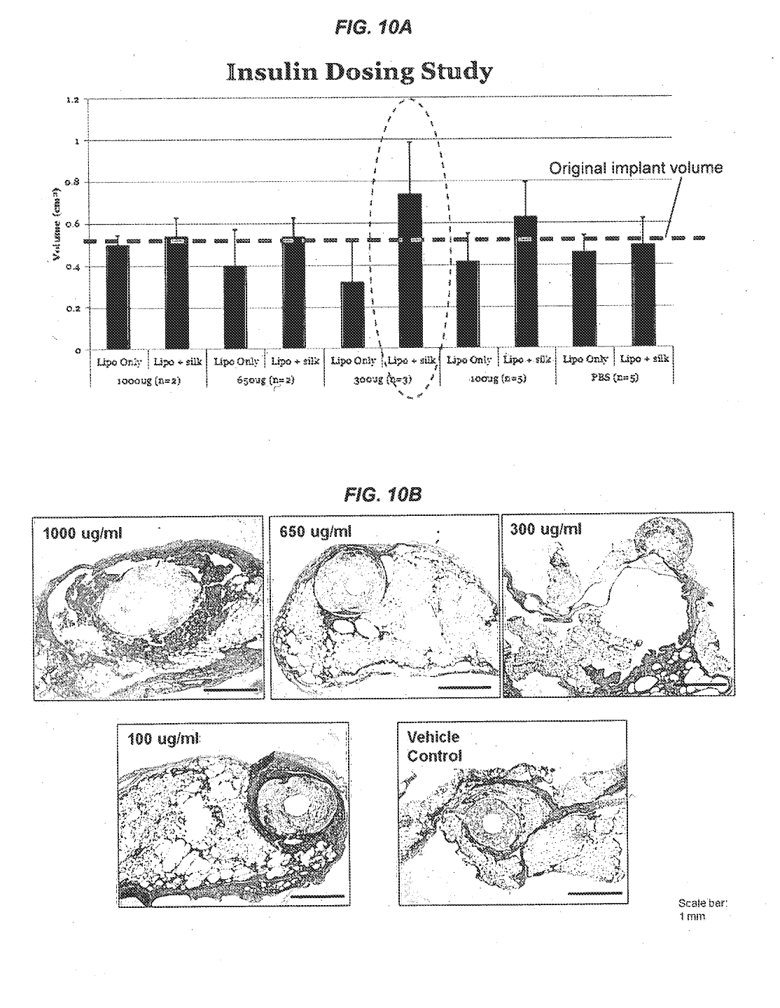

The volumes of the harvested fat grafts were measured using gas pycnometry. For the dexamethasone treated animals, there were no statistically significant differences detected, however the largest effect was found with the ˜2 mg/ml dose (FIG. 9A). The grafts with no fibrous tissue did not appear to show a significant effect between the groups, which indicate that the lipoaspirate-only controls were not affected by the release of dexamethasone into the fat graft containing the silk fibroin-based catheter. H&E images (FIG. 9B) show little to no cellular response to the silk fibroin-based catheter placement (outlined with dashed circles when appropriate) with the vehicle control, and the ˜1 and ˜2 mg/ml doses of dexamethasone. There appeared to be a mild inflammatory response in the area immediately surrounding the fiber at the 3 mg/ml dose (FIG. 9B). For the insulin-treated animals, there was no statistical significance in volume retention; however the group with the highest volume retention was ˜300 μg (no deaths due to drug overdose, but still showed a trend toward fat retention and adipogenesis) (FIG. 10A). H&E images show little to no cellular response to the silk fibroin-based catheter with the insulin doses and vehicle control (FIG. 10B).

All patents and other publications identified in the specification and examples are expressly incorporated herein by reference for all purposes. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

What is claimed is:

1. A system comprising:
a perfusion device; and
a hollow tube extending along a longitudinal axis from a first end to a second end; wherein the hollow tube is formed of a material comprising silk fibroin, and
wherein the first end is adaptably connected to the perfusion device.

2. The system of claim 1, wherein the perfusion device further comprises a chamber configured to store a fluid to be delivered by the perfusion device into the hollow tube.

3. The system of claim 1 or 2, wherein the hollow tube is adapted to permit passage of the fluid to be released across at least a portion of a wall forming the hollow tube.

4. The system of claim 3, wherein the at least a portion of the wall forming the hollow tube is porous.

5. The system of any of claims 2-4, wherein the fluid comprises at least one agent.

6. The system of claim 5, wherein the agent is selected from the group consisting of cells; therapeutic agents; small organic or inorganic molecules; saccharides; oligosaccharides; polysaccharides; biological macromolecules, e.g., peptides, proteins, and peptide analogs and derivatives; peptidomimetics; antibodies and antigen binding fragments thereof; nucleic acids; nucleic acid analogs and derivatives; glycogens or other sugars; immunogens; antigens; an extract made from biological materials such as bacteria, plants, fungi, or animal cells; animal tissues; naturally occurring or synthetic compositions; and any combinations thereof.

7. The system of claim 5 or 6, wherein the agent comprises an adipogenic agent.

8. The system of claim 7, wherein the adipogenic agent comprises insulin and/or dexamethasone.

9. The system of any of claims 5-8, wherein the agent comprises an angiogenic agent.

10. The system of any of claims 1-9, wherein the second end of the hollow tube is sealed.

11. The system of claim 10, wherein the second end of the hollow tube is sealed with silk fibroin.

12. The system of any of claims 1-11, wherein the perfusion device is an extracorporeal device.

13. The system of any of claims 1-11, wherein the perfusion device is an implantable device.

14. The system of any of claims 1-13, wherein the system is implantable.

15. The system of any of claims 1-14, wherein the first end is adaptably connected to the perfusion device using a sealant.

16. The system of any of claims 1-15, wherein the inside diameters of the hollow tube at the first end and at the second end differ by at least about 5%.

17. An ex vivo system comprising:
a hollow tube extending along a longitudinal axis from a first end to a second end; wherein the hollow tube is formed of a material comprising silk fibroin, and at least one end is adapted to be capable of connecting to a connector and/or a device; and
a tissue comprising at least a portion of the hollow tube disposed therein.

18. The ex vivo system of claim 17, wherein the tissue is derived from a subject.

19. The ex vivo system of claim 17, wherein the tissue is a tissue scaffold comprising cells.

20. The ex vivo system of any of claims 17-19, further comprising the connector and/or device adaptably connected to said at least one end.

21. The ex vivo system of any of claims 17-20, wherein the connector and/or device comprises a fluid delivery tubing, a cannula, an adaptor, a perfusion device, or any combinations thereof.

22. The ex vivo system of claim 21, wherein the perfusion device further comprises a chamber configured to store a fluid to be delivered by the perfusion device into the hollow tube.

23. The ex vivo system of any of claims 17-22, wherein the hollow tube is adapted to permit passage of the fluid to be released across at least a portion of a wall forming the hollow tube.

24. The ex vivo system of claim 23, wherein the at least a portion of the wall forming the hollow tube is porous.

25. The ex vivo system of any of claims 22-24, wherein the fluid comprises at least one agent.

26. The ex vivo system of claim 25, wherein the agent is selected from the group consisting of cells; therapeutic agents; small organic or inorganic molecules; saccharides; oligosaccharides; polysaccharides; biological macromolecules, e.g., peptides, proteins, and peptide analogs and derivatives; peptidomimetics; antibodies and antigen binding fragments thereof; nucleic acids; nucleic acid analogs and derivatives; glycogens or other sugars; immunogens; antigens; an extract made from biological materials such as bacteria, plants, fungi, or animal cells; animal tissues; naturally occurring or synthetic compositions; and any combinations thereof.

27. The ex vivo system of claim 25 or 26, wherein the agent comprises an adipogenic agent.

28. The ex vivo system of claim 27, wherein the adipogenic agent includes insulin and/or dexamethasone.

29. The ex vivo system of any of claims 25-28, wherein the agent comprises an angiogenic agent.

30. The ex vivo system of any of claims 17-29, wherein the end of the hollow tube that is not connected to the connector and/or device is sealed.

31. The ex vivo system of claim 30, wherein the end of the hollow tube that is not connected to the connector and/or device is sealed with silk fibroin.

32. The ex vivo system of any of claims 17-31, wherein the connector and/or device is adapted for connection outside of the tissue.

33. The ex vivo system of any of claims 17-31, wherein the connector and/or device is adapted for implantation in the tissue.

34. The ex vivo system of any of claims 17-33, wherein the inside diameters of the hollow tube at the first end and at the second end differ by at least about 5%.

35. A biopolymer device comprising:
a hollow tube extending along a longitudinal axis from a first end to a second end, wherein the hollow tube is formed of a material comprising silk fibroin; and wherein the first end is adaptably connected to a connector and/or a device.

36. The biopolymer device of claim 35, wherein the connector and/or device includes a fluid delivery tubing, a cannula, an adaptor, a pump, or any combinations thereof.

37. The biopolymer device of claim 36, wherein the fluid delivery tubing, cannula, or adaptor is made of a material comprising polypropylene.

38. The biopolymer device of any of claims 35-37, wherein the connector and/or device is adapted for extracorporeal or implantation use.

39. The biopolymer device of any of claims 35-37, wherein the biopolymer device is adapted to be implantable.

40. The biopolymer device of any of claims 35-39, wherein the inside diameter of the hollow tube at the first end is different from the inside diameter of the hollow tube at the second end.

41. The biopolymer device of claim 40, wherein the inside diameter of the hollow tube at the first end adaptably connected to the connector and/or device is larger than the inside diameter of the hollow tube at the second end.

42. A biopolymer device comprising:
a hollow tube extending along a longitudinal axis from a first end to a second end, the inside diameter of the hollow tube at the first end being larger than the inside diameter of the hollow tube at the second end; and
wherein the hollow tube is formed of a material comprising silk fibroin.

43. The biopolymer device of claim 42, wherein the inside diameter of the hollow tube at the first end is larger than the inside diameter of the hollow tube at the second end by at least about 5%.

44. The biopolymer device of claim 42 or 43, wherein the second end includes a seal or a cap.

45. The biopolymer device of claim 44, wherein the seal or cap comprises silk fibroin.

46. The biopolymer device of any of claims 42-45, wherein the hollow tube is adapted to permit passage of a fluid to be released across at least a portion of a wall forming the hollow tube.

47. The biopolymer device of claim 46, wherein the at least a portion of the wall forming the hollow tube is porous.

48. The biopolymer device of any of claims 42-47, wherein the first end or the second end is adapted to be capable of connecting to a connector and/or a device.

49. The biopolymer device of claim 48, further comprising the connector and/or the device adaptably connected to the first end or the second end.

50. The biopolymer device of claim 48 or 49, wherein the connector and/or device includes a fluid delivery tubing, a cannula, an adaptor, a perfusion device, a pump, or any combinations thereof.

51. The biopolymer device of claim 50, wherein the perfusion device is a perfusion pump.

52. The biopolymer device of claim 51, wherein the perfusion device further comprises a chamber configured to store a fluid to be introduced into the hollow tube.

53. The biopolymer device of claim 52, wherein the fluid further comprises an agent.

54. The biopolymer device of claim 53, wherein the agent is selected from the group consisting of cells; therapeutic agents; small organic or inorganic molecules; saccharides; oligosaccharides; polysaccharides; biological macromolecules, e.g., peptides, proteins, and peptide analogs and derivatives; peptidomimetics; antibodies and antigen binding fragments thereof; nucleic acids; nucleic acid analogs and derivatives; glycogens or other sugars; immunogens; antigens; an extract made from biological materials such as bacteria, plants, fungi, or animal cells; animal tissues; naturally occurring or synthetic compositions; and any combinations thereof.

55. The biopolymer device of claim 53 or 54, wherein the agent comprises an adipogenic agent.

56. The biopolymer device of claim 55, wherein the adipogenic agent includes insulin and/or dexamethasone.

57. The biopolymer device of claim 53 or 54, wherein the agent comprises an angiogenic agent.

58. The biopolymer device of any of claims 42-57, wherein the hollow tube is flexible.

59. The biopolymer device of any of claims 42-58, wherein the hollow tube degrades over a period of time upon implantation in vivo.

60. The biopolymer device of any of claims 42-59, wherein the biopolymer device is adapted to be a catheter.

61. The biopolymer device of any claims 42-59, wherein the biopolymer device is adapted to be a stent.

62. A method comprising:
placing at least a portion of the hollow tube of the system of any of claims 1-16, or of the ex vivo system of any of claims 17-34, or of the biopolymer device of any of claims 35-61 at a target site.

63. The method of claim 62, wherein the target site is a tissue to be perfused with a fluid delivered by the system, the ex vivo system or the biopolymer device.

64. The method of claim 62 or 63, wherein the target site is a tissue to be repaired or regenerated.

65. The method of claim 63 or 64, wherein the tissue is ex vivo.

66. The method of claim 63 or 64, wherein the tissue is present in a subject.

67. The method of any of claims 62-66, further comprising directing a fluid into the hollow tube, wherein the fluid optionally comprises an agent.

68. The method of claim 67, wherein the fluid permeates across at least a portion of the wall of the hollow tube to the target site over a period of time.

69. The method of any of claims 62-68, further comprising introducing cells at the target site.

70. The method of claim 69, wherein the introduced cells surround at least a portion of the hollow tube.

71. The method of claim 69, wherein at least 50% of the introduced cell volume is retained at the target site over the period of time.

72. The method of any of claims 62-71, further comprising growing cells surrounding the hollow tube.

73. The method of any of claims 62-72, wherein the hollow tube degrades as a tissue surrounding the target site remodels or regenerates.

74. A method for repairing or regenerating a tissue, the method comprising
implanting at least a portion of the hollow tube of the system of any of claims 1-16, or of the ex vivo system of any of claims 17-34, or of the biopolymer device of any of claims 35-61, in a tissue to be repaired or regenerated, wherein the tissue is perfused with a fluid delivered by the system, ex vivo system or biopolymer device, thereby promoting repair or regeneration of the tissue.

75. The method of claim 74, wherein the tissue is ex vivo.

76. The method of claim 74, wherein the tissue is present in a subject.

77. The method of any of claims 74-76, further introducing cells into the tissue.

78. The method of claim 77, wherein the cells are derived from lipoaspirate.

79. The method of claim 77 or 78, wherein the cells are delivered via the system, ex vivo system or biopolymer device.

80. The method of any of claims 77-79, wherein the cells are implanted in the tissue prior to, concurrently with, or after the implantation of the hollow tube.

81. The method of any of claims 74-80, further comprising introducing the fluid into the hollow tube.

82. The method of claim 81, wherein the fluid permeates across at least a portion of a wall forming the hollow tube.

83. The method of any of claims 74-82, wherein the fluid comprises an agent.

84. The method of claim 83, wherein the agent comprises an adipogenic agent, an angiogenic agent, or a combination thereof.

85. The method of any of claims 74-84, wherein the tissue is a soft tissue.
